SEQUENCE LISTING (18 sequences)

SEQ ID NO: 1 - DNA, 21 bp, Artificial Sequence
  Target Sequence for RNAi
gactacgagcagatcaagata

SEQ ID NO: 2 - DNA, 21 bp, Artificial Sequence
  Target Sequence for RNAi
agaaagagaagcatccgaaat

SEQ ID NO: 3 - DNA, 58 bp, Artificial Sequence
  shRNA Sequence
ccggcccggacgagctgacggtcaactcgagttgaccgtcagctcgtccgggtttttg

SEQ ID NO: 4 - DNA, 58 bp, Artificial Sequence
  shRNA Sequence
aattcaaaaacccggacgagctgacggtcaactcgagttgaccgtcagctcgtccggg

SEQ ID NO: 5 - DNA, 58 bp, Artificial Sequence
  shRNA Sequence
ccgggatcaccatcccagtcaccttctcgagaaggtgactgggatggtgatctttttg

SEQ ID NO: 6 - DNA, 58 bp, Artificial Sequence
  shRNA Sequence
aattcaaaaagatcaccatcccagtcaccttctcgagaaggtgactgggatggtgatc

SEQ ID NO: 7 - DNA, 58 bp, Artificial Sequence
  shRNA Sequence
ccggggagctgttggtgggaatatactcgagtatattcccaccaacagctcctttttg

SEQ ID NO: 8 - DNA, 58 bp, Artificial Sequence
  shRNA Sequence
aattcaaaaaggagctgttggtgggaatatactcgagtatattcccaccaacagctcc

SEQ ID NO: 9 - DNA, 20 bp, Artificial Sequence
  synthetic primer
ggagaagggtgaccgactca

SEQ ID NO: 10 - DNA, 17 bp, Artificial Sequence
  synthetic primer
ctgcccagactcggcaa

SEQ ID NO: 11 - DNA, 20 bp, Artificial Sequence
  synthetic primer
tacacctcctccttctgggg

SEQ ID NO: 12 - DNA, 20 bp, Artificial Sequence
  synthetic primer
tccaatgaggtgagcagcag

SEQ ID NO: 13 - DNA, 19 bp, Artificial Sequence
  synthetic primer
caccttttagcttcctgac

SEQ ID NO: 14 - DNA, 20 bp, Artificial Sequence
  synthetic primer
aatttcactgcttcatccag

SEQ ID NO: 15 - DNA, 20 bp, Artificial Sequence
  synthetic primer
ctaaacagatgaagtgctcc

SEQ ID NO: 16 - DNA, 18 bp, Artificial Sequence
  synthetic primer
ggtcattctcctggaagg

SEQ ID NO: 17 - DNA, 20 bp, Artificial Sequence
  synthetic primer
cccacaacgaaatctatgac

SEQ ID NO: 18 - DNA, 20 bp, Artificial Sequence
  synthetic primer
tgtatttctggtacagctcc

(19) United States
(12) Patent Application Publication
(10) Pub. No.: US 2023/0087078 A1
(43) Pub. Date: Mar. 23, 2023

(54) COMPOSITIONS AND METHODS FOR THE TREATMENT OF PANCREATIC CANCER

(71) Applicant: Washington University, St. Louis, MO (US)

(72) Inventors: Kian-Huat Lim, St. Louis, MO (US); Patrick Grierson, St. Louis, MO (US)

(73) Assignee: Washington University, St. Louis, MO (US)

(21) Appl. No.: 17/908,863

(22) PCT Filed: Mar. 2, 2021
(86) PCT No.: PCT/US21/20538

§ 371 Date: Sep. 1, 2022

Related U.S. Application Data
(60) Provisional application No. 62/983,776, filed on Mar. 2, 2020.

(51) Int. Cl. A61K 31/551; A61K 45/06; A61K 31/506; A61K 31/437; A61P 35/00

(52) U.S. Cl. CPC A61K 31/551; A61K 45/06; A61K 31/506; A61K 31/437; A61P 35/00

(57) ABSTRACT

The present disclosure describes a method for the treatment of pancreatic cancer includes administering an effective amount of an MK2 inhibiting agent. In other aspects, the method may include administering the effective amount of the MK2 inhibiting agent in combination with a chemotherapy composition. In additional aspects, a composition for the treatment of pancreatic cancer is disclosed that includes an effective amount of an MK2 inhibiting agent and an effective amount of a chemotherapy composition.

### CROSS-REFERENCE TO RELATED APPLICATIONS

This application claims the benefit of priority to U.S. Provisional Application No. 62/983,776, filed on Mar. 2, 2020, the content of which is incorporated by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE DISCLOSURE

The present disclosure generally relates to compositions and methods of treatment of pancreatic cancer.

### BACKGROUND OF THE DISCLOSURE

Pancreatic ductal adenocarcinoma (PDAC) is emerging as one of the leading causes of cancer-related death in the US, highlighting the urgency to develop more effective therapeutic strategies. To date, combination chemotherapies remain the only effective systemic PDAC treatment. Existing molecular-targeted and immuno-therapies have not been successful to date, although existing combination chemotherapies, particularly FOLFIRINOX (a cocktail of folinic acid, 5-FU, irinotecan, and oxaliplatin) and gemcitabine/nab-paclitaxel do significantly and meaningfully prolong the survival of PDAC patients. For patients with early-stage PDAC who undergo surgical resection, adjuvant FOLFIRINOX cures an additional ˜20% of patients compared to gemcitabine. These results indicate that incurring extensive, irreparable DNA damage is an effective strategy in PDAC cells. However, these regimens are clinically toxic, and less than one-third of treated patients achieve an objective radiographic response to either regimen, underscoring the need to overcome de novo resistance mechanisms in order to augment treatment response. Because gemcitabine monotherapy was previously the standard treatment regimen, most literature has focused on identifying resistance mechanisms to gemcitabine, and resistance mechanisms to FOLFIRINOX are less studied.

Depending on the extent of DNA damage, cells invoke distinct sets of signaling networks that determine the ultimate cell fate. In general, DNA damage is first sensed by DNA-damage response (DDR) signaling pathways, typified by the ATM/ATR-CHK1/CHK2 axis, leading to cell cycle arrest, during which DNA repair via various mechanisms including non-homologous end-joining, homologous recombination, mismatch repair, and nucleotide excision repair, are engaged. To facilitate DNA repair, various pro-survival mechanisms must be engaged including activation of the NF-κB, p38MAPK, and AKT pathways, which heighten the survival threshold, as well as autophagy that enables scavenging of macromolecule precursors. Simultaneously, DDR also triggers various death mechanisms, but these are countered by survival mechanisms, pending the outcome of DNA repair. Cells that successfully repair their DNA or become tolerant of unrepaired DNA lesions will survive, whereas those that fail to repair their DNA damage will die by apoptosis or necrosis.

In PDAC treatment, escalating the strength of chemotherapy is unlikely to be tolerable. Therefore, lowering the survival threshold of PDAC cells, such as by targeting the pro-survival NF-κB or AKT pathways, deprives these cells of the opportunity for DNA repair. However, the addition of the PI3K inhibitor rigosertib or the autophagy inhibitor hydroxychloroquine failed to improved survival for PDAC patients treated with gemcitabine-based regimens, prompting the need to develop novel approaches.

Targeting the canonical MAPK cascade is a heavily pursued therapeutic strategy in pancreatic ductal adenocarcinomas (PDAC), as almost all cases harbor activating mutations in the KRAS or BRAF genes. Clinical success remains limited due to de novo resistance arising from cell-intrinsic pathway rewiring and poor drug delivery due to the hypovascular and fibrotic extrinsic tumor micro-environment (TME) unique to PDAC tumors. The Toll-like receptor signaling driven by IRAK4 has been demonstrated to be critical in conferring PDAC cell survival and tumor fibrosis. Constitutive IRAK4 activation drives multiple signaling pathways including the IKK_NF-kB, p38/MK2, and the IRF-IFN pathways. The IKK_NF-kB cascade has been shown to drive tumor fibrosis and chemoresistance. However, a clinical-grade IKK inhibitor is not available. The role of the p38/MK2 pathway in PDAC progression and growth has not been thoroughly investigated. Understanding this aspect of PDAC development and progression may inform the development of novel therapeutic strategies.

Other objects and features will be in part apparent and in part pointed out hereinafter.

### SUMMARY OF THE DISCLOSURE

In one aspect, a composition for the treatment of pancreatic cancer in a subject in need is disclosed that includes an effective amount of an MK2 inhibiting agent and an effective amount of a chemotherapy composition. In some aspects, the MK2 inhibiting agent is selected from a small molecule, an interfering protein, an antibody, an shRNA, and an siRNA. In some aspects, the MK2 inhibiting agent targets MK2 or MK2R1. In some aspects, the MK2 inhibiting agent is PF-3644022 or ATI-450. In some aspects, the chemotherapy composition comprises irinotecan. In some aspects, the chemotherapy composition includes at least one of leucovorin calcium (folinic acid), fluorouracil (5-FU), irinotecan, oxaliplatin, gemcitabine, nab-paclitaxel, and any combination thereof. In some aspects, the chemotherapy composition is selected from FOLFIRINOX, FOLFOX, FOLFOX/CD40 agonist/anti-PD1 cocktail, FOLFOX/anti-CTLA4/anti-PD1 cocktail, and gemcitabine/nab-paclitaxel.

In another aspect, a method for treating pancreatic cancer in a subject in need is disclosed that includes administering an effective amount of an MK2 inhibiting agent to the subject. In some aspects, the method further includes administering an effective amount of a chemotherapy composition in combination with the effective amount of the MK2 inhibiting agent. In some aspects, the MK2 inhibiting agent is selected from a small molecule, an interfering protein, an antibody, an shRNA, and a siRNA. In some aspects, the MK2 inhibiting agent targets MK2 or MK2R1. In some aspects, the MK2 inhibiting agent is PF-3644022 or ATI-450. In some aspects, the chemotherapy composition comprises irinotecan. In some aspects, the chemotherapy composition comprises at least one of leucovorin calcium (folinic acid), fluorouracil (5-FU), irinotecan, oxaliplatin, gemcitabine, nab-paclitaxel, and any combination thereof In some aspects, the chemotherapy composition is selected from FOLFIRINOX, FOLFOX, FOLFOX/CD40 agonist/anti-PD1 cocktail, FOLFOX/anti-CTLA4/anti-PD1 cocktail, and gemcitabine/nab-paclitaxel. In some aspects, the pancreatic cancer is pancreatic ductal adenocarcinoma.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A contains a heat map of RPPA showing significantly upregulated and downregulated proteins in Pa01c cells following treatment with FIRINOX (right) and a vehicle treatment (right).

FIG. 1B is a graph summarizing the log 2-fold changes of the most significantly upregulated and downregulated proteins in Pa01c cells following treatment with FIRINOX from the heat map of FIG. 1A.

FIG. 1C contains images of contains images from Western blot analysis confirming upregulated changes in Pa01c cells identified by RPPA.

FIG. 1D contains immunoblot images showing the degree of PARP cleavage in control Pa01 cells treated with FOLFIRINOX alone or with JNKi or MEKi, and Pa01c cells stably expressing Hsp27 shRNA treated with or without FIRINOX.

FIG. 1E contains immunoblot images showing the degree of PARP cleavage in MIA paca-2 vector control cells or MIA paca-2 cells stably expressing Hsp27 shRNA, and treated with control or the individual components of FOLFIRINOX (5-FU, SN38, oxaliplatin).

FIG. 1F contains scatter plots (left) and a quantification graph (right) summarizing a. FACS quantification of cells as treated in FIG. 1E, with analysis limited to SN38 treated cells.

FIG. 2A contains immunoblot images showing upregulation of p-Hsp27 and p-MK2 in a panel of PDAC cell lines treated with the individual components of FIRINOX (5-FU, SN38, oxaliplatin) or gemcitabine.

FIG. 2B contains immunoblot images showing MK2 dependence of p-Hsp27 induction in Pa01c vector control or stable MK2 shRNA expressing cells treated with control or SN38.

FIG. 2C contains immunoblot images showing PARP cleavage in MIA paca-2 vector control or stable MK2 shRNA expressing cells treated with the individual components of FIRINOX or gemcitabine.

FIG. 2D contains scatter plots (left) and a quantification graph (right) summarizing a FACS analysis showing Annexin V and PI staining of MIA paca-2 cells treated with control, PF-3644022, SN38, or the combination. P values from one-way ANOVA with Dunnett's multiple comparison test and error bars are mean+SEM. **P<0.0021, ****P<0.0001.

FIG. 2E contains immunoblot images showing PARP cleavage of MIA paca-2 and Pa01c cells treated as in FIG. 1D.

FIG. 2F contains scatter plots (left) and a quantification graph (right) summarizing a FACS analysis showing Annexin V and PI staining of MIA paca-2 cells treated with control, ATI-450, SN38, or the combination.

FIG. 2G contains immunoblot images showing PARP cleavage of MIA paca-2 and Pa01c cells treated as in FIG. 1F.

FIG. 2H contains immunoblot images of p-p38, p-MK2, and p-Hsp27 from MIA paca-2 and Pa01c cells treated with 40 J/m2 of ultraviolet light (UV) alone or with MK2 inhibitor PF-3644022.

FIG. 2I contains immunoblot images of p-MK2 and p-Hsp27 from Pa01c cells treated with 40 J/m2 UV light, alone or with MK2 inhibitor ATI-450.

FIG. 3A contains immunoblot images showing p-p38, p-MK2, and p-Hsp27 activation in MIA paca-2 and Pa01c cells treated with SN38 10 μM for 16 hours alone or with IRAK4 inhibitor 4 μM, HCQ 20 μM, ATM inhibitor 20 μM, ATR inhibitor 20 μM, or TAK1 inhibitor 2 μM.

FIG. 3B contains immunoblot images of MIA paca-2 and Pa01c cells treated with SN38 10 μM alone for 16 hours or in combination with TAK1 inhibitor 2 μM for 16 hours.

FIG. 3C contains immunoblot images of cells over-expressing FLAG-tagged TAK1 co-immunoprecipitation.

FIG. 3D contains graphs summarizing the results of qPCR for TNFα, TNFβ, TGFβ, IL-1α, and IL-1β in Mia paca-2 (right) and Pa01c (left) cells treated with control or SN38 10 μM for 16 hours. P values from two-away ANOVA with Dunnett's multiple comparison test and error bars are mean+SEM. ***P<0.0002, ****P<0.0001.

FIG. 3E is a graph summarizing TNFα ELISA of Pa01c cells treated with control or SN38 10 μM for 16 hours overnight. P values from one-way ANOVA with Dunnett's multiple comparison test and error bars are mean+SEM. *P<0.0332.

FIG. 3F is a graph summarizing the results of qPCR showing TNFα expression in Pa01c cells following treatment with SN38 10 μM alone for 16 hours, or in combination with TAKli 2 μM or IKKi 2 μM for 24 hours prior to treatment with SN38 10 μM for 16 hours. P values from two-away ANOVA with Dunnett's multiple comparison test and error bars are mean+SEM. *P<0.0247, **P<0.0025.

FIG. 3G contains immunoblot images of MIA paca-2 and Pa01c cells treated with control, TNFα 10 ng/ml, or SN38 10 μM for 16 hours.

FIG. 3H contains immunoblot images showing PARP cleavage in MIA paca-2 and Pa01c cells treated with control, TNFα 10 ng/ml or TNFα 10 ng/ml plus ATI-450 5 μM.

FIG. 3I contains scatter plots (left) and a graph summarizing cell viability quantification via FACS analysis of MIA paca-2 cells treated with control, ATI-450 5 μM, TNFα 40 ng/ml, or the combination for 36 hours. P values from one-way ANOVA with Tukey's multiple comparison test and error bars are mean+SEM. *P<0.0285, ****P<0.0001.

FIG. 3J contains immunoblot images showing p-Hsp27 and PARP cleavage of MIA paca-2 and Pa01c cells stably expressing scrambled shRNA or TNF receptor (TNFR) shRNA treated with control or SN38 10 μM for 16 hours overnight.

FIG. 4A is a graph summarizing the quantification of RPPA showing fold change of p-AMPK T172 and p-ULK1 5757 in Pa01c cells following FOLFIRINOX treatment. P values from two-way ANOVA with Sidak's multiple comparison test and error bars are mean+SEM. **P<0.0085.

FIG. 4B is an image of immunoblots of proteins mediating autophagy in MIA paca-2 and Pa01c cells following treatment with gemcitabine, FOLFIRINOX, or the components of FOLFIRINOX (5-FU, SN38, oxaliplatin).

FIG. 4C is a graph summarizing fluorescence analysis of Pa01c and MIA paca-2 cells transfected with mCherry-EGFP-LC3 construct following treatment with SN38 10 μM or DMSO for 24 hours. P values from two-way ANOVA with Sidak's multiple comparison test and error bars are mean+SEM. ****P<0.0001.

FIG. 4D contains immunoblot images of MIA paca-2 and Pa01c cells following treatment with SN38, HCQ, or the combination. P values from two-way ANOVA with Sidak's multiple comparison test and error bars are mean+SEM. ***P=0.0009, *P=0.035.

FIG. 4E contains immunoblot images of MIA paca-2 and Pa01c cells showing markers of autophagy following treatment with MK2 inhibitors (ATI-450 or PF-35644022), SN38, or the combination.

FIG. 4F contains immunoblot images of MIA paca-2 control cells or Beclin 1 (BECN1) knockout treated with SN38, ATI-450, or the combination.

FIG. 4G contains scatter plots (left) and a graph summarizing quantification of a FACS analysis of MIA paca-2 control cells or Beclin 1 knockout cells treated with control (DMSO) or SN38 10 μM for 24 hours; graphic quantification of annexin V positive cells on the right.

FIG. 5A contains an image of immunoblots showing p-p38, p-MK2, and p-Hsp27 across a panel of PDAC cell lines.

FIG. 5B contains immunohistochemistry (IHC) images of pancreatic tumors from KPC mice and human subjects showing p38 and MK2 activation status in normal epithelium, PanIN, and adenocarcinoma.

FIG. 5C contains graphs of tumor growth curves of MIA paca-2, Pa01c, and AsPc-1 cells stably expressing scrambled shRNA or MK2 shRNA injected subcutaneously into nude mice.

FIG. 5D contains a series of immunohistochemistry (IHC) images of spontaneously forming tumors in KPPC mice treated with control or ATI-450 chow (top), scored for abundance of normal epithelium, early PanIN, late PanIN, PDAC, or necrosis (bottom).

FIG. 5E contains images of Sirius red staining of PDAC tumors from KPPC mice treated with control or ATI-450 chow (left) and a graph comparing the quantified Sirius red areas of the images (right).

FIG. 6A is a graph comparing tumor growth curves of wild type MIA paca-2 cells injected subcutaneously into nude mice and treated with vehicle, ATI-450, FOLFIRINOX, or ATI-450 plus FOLFIRINOX.

FIG. 6B is a graph comparing tumor growth curves of wild type Pa01c cells injected subcutaneously into nude mice and treated with vehicle, ATI-450, FOLFIRINOX, or ATI-450 plus FOLFIRINOX.

FIG. 6C is a graph comparing Kaplan-Meier survival curves of the KPPC mice treated with vehicle, ATI-450, FOLFIRINOX, or ATI-450 plus FOLFIRINOX.

FIG. 6D is a graph summarizing final tumor weights from the KPPC mice shown in FIG. 6C at the time of sacrifice.

FIG. 6E contains a series of H&E images (left column), images stained for p-Hsp27 (center column), and images stained for p-MK2 (right column) on tumors derived from mice of FIG. 6C.

FIG. 6F contain H&E and Sirius Red images of tumors derived from mice treated with vehicle or ATI-450 plus FOLFIRINOX in FIG. 6C.

FIG. 6G contains a series of immunofluorescence images for pan-cytokeratin (pan-CK) and cleaved caspase 7 (CC7) on tumors derived from the KPPC mice of FIG. 6C, as well as a graph (right) summarizing activity levels derived from the immunofluorescence images.

FIG. 6H contains a series of immunohistochemistry (IHC) images for p-MK2 on human PDAC tumor tissue microarray (TMA) derived from patients undergoing pancreatectomy followed by adjuvant chemotherapy (left) and a graph summarizing a Kaplan-Meier survival analysis based on p-MK2 levels (right).

FIG. 7 is a flow chart illustrating the modulation of pancreatic cancer cell survival by the disclosed TAK1/MK2/Hsp27 axis.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE INVENTION

In various aspects, compositions and methods for the treatment of pancreatic cancers including, but not limited to pancreatic ductal adenocarcinomas (PDAC), are disclosed herein. The disclosed compositions and methods are based on the discovery of MK2 as a novel therapeutic target that can greatly enhance the therapeutic efficacy of chemotherapy in pancreatic cancer. In some aspects, an MK2 inhibitor compound including, but not limited to ATI-450, may be administered to a patient in need in combination with standard chemotherapy. As illustrated in the examples provided below, administration of an MK2 inhibitor in combination with standard chemotherapy is highly effective and eradicated pancreatic cancer in the most aggressive pancreatic cancer mouse model.

The disclosed compositions and methods provide a clear oncologic regimen that can be readily tested in clinical trials for patients with cancers commonly treated with chemotherapy, including pancreatic cancer. The disclosed combination regimen could improve the survival of treated patients.

In some aspects, a method for the treatment of pancreatic cancer includes administering an MK2 inhibitor. In other aspects, a method for the treatment of pancreatic cancer includes administering an MK2 inhibitor in combination with a chemotherapy composition. In various aspects, the chemotherapy composition may include at least one chemotherapy compound. The chemotherapy composition may include one chemotherapy compound, two chemotherapy compounds, three chemotherapy compounds, three chemotherapy compounds, four chemotherapy compounds, five chemotherapy compounds, or more. Non-limiting examples of suitable chemotherapy compounds include leucovorin calcium (folinic acid), fluorouracil (5-FU), irinotecan, oxaliplatin, gemcitabine, nab-paclitaxel, and any combination thereof. Non-limiting examples of irinotecan-containing chemotherapy compositions include but not limited to, FOLFIRINOX (a cocktail of folinic acid, 5-FU, irinotecan, and oxaliplatin), FOLFOX (a cocktail of leucovorin calcium (folinic acid), fluorouracil, and oxaliplatin), FOLFOX/CD40 agonist/anti-PD1 cocktail, FOLFOX/anti-CTLA4/anti-PD1 cocktail, and gemcitabine/nab-paclitaxel.

In various aspects, the chemotherapy composition may include a method for the treatment of pancreatic cancer includes administering an MK2 inhibitor in combination with an irinotecan-containing chemotherapy composition. Without being limited to any particular theory, suppression of kinase MAPKAPK2 (or MK2) by a small molecule inhibitor or RNAi has been demonstrated to significantly augment the apoptotic effect of irinotecan-containing chemotherapy treatments. Mechanistically, SN38 (the active metabolite of irinotecan) dramatically upregulates TNFα, leading to TNFα-dependent autocrine phosphorylation of TAK1, MK2, and Hsp27. Suppression of MK2 abrogates Hsp27 activation, sensitizes cells to apoptosis, and additionally suppresses SN38-induced protective autophagy, in part by suppressing phosphorylation of Beclin-1. Non-limiting examples of chemotherapy compositions include, but are not limited to, FOLFIRINOX (a cocktail of folinic acid, 5-FU, irinotecan, and oxaliplatin).

In various aspects, the disclosed compositions and methods are suitable for the treatment of pancreatic cancers. Although the disclosure and examples disclosed herein are directed to the treatment of pancreatic ductal adenocarcinomas (PDAC), the compositions and methods are suitable for the treatment of any pancreatic cancer without limitation in various aspects. Non-limiting examples of pancreatic cancer types suitable for treatment using the disclosed compositions and methods include exocrine pancreatic cancer and endocrine or neuroendocrine pancreatic cancer. Non-limiting examples of exocrine pancreatic cancers include adenocarcinomas, acinar cell carcinomas, intraductal papillary-mucinous neoplasms (IPMNs), and mucinous cystic neoplasms with invasive adenocarcinomas. The term “pancreatic ductal adenocarcinomas (PDAC)”, as used herein, refers to the adenocarcinoma type of exocrine/ductal pancreatic cancer. Non-limiting examples of neuroendocrine pancreatic cancers, also known as endocrine pancreatic cancers, include pancreatic neuroendocrine tumors (NETs), also known as endocrine or islet cell tumors. Non-limiting examples of types of neuroendocrine tumors (NETs), as defined by the endocrine activity of the affected cells, include gastrinomas (gastrin-producing cells), glucaganomas (glucagon-producing cells), insulinomas (insulin-producing cells), somatostatinomas (somatostatin-producing cells), VIPomas (vasoactive intestinal peptide-producing cells), and nonfunctional Islet Cell Tumor (cells with no hormonal activity).

### MK2 Inhibiting Agents

One aspect of the present disclosure provides for targeting of MK2 or its downstream signaling. The present disclosure provides methods of treating or preventing pancreatic cancer based on the discovery that silencing of Hsp27 by RNA interference (RNAi), or suppression of its upstream kinase MAPKAPK2 (or MK2) by small molecule inhibitor or RNAi, significantly augmented the apoptotic effect of various chemotherapy regimens.

As described herein, inhibitors of MK2 (e.g., antibodies, fusion proteins, small molecules) can reduce or prevent pancreatic cancer. An MK2 inhibiting agent can be any agent that can inhibit MK2, downregulate MK2, or knockdown MK2.

As an example, an MK2 inhibiting agent can inhibit heat-shock protein 27 (Hsp27) signaling.

For example, the MK2 inhibiting agent can be an anti MK2 antibody. Furthermore, the anti-MK2 antibody can be a murine antibody, a humanized murine antibody, or a human antibody.

As another example, an MK2 inhibiting agent can be PF-3644022 or ATI-450, which are potent and specific inhibitors of MK2 signaling.

As another example, an MK2 inhibiting agent can be an inhibitory protein that antagonizes MK2 activation or downstream signaling. For example, the MK2 inhibiting agent can be a viral protein, which has been shown to antagonize MK2 activation or downstream signaling.

As another example, an MK2 inhibiting agent can be a short hairpin RNA (shRNA) or a short interfering RNA (siRNA) targeting MK2 or MK2R1.

As another example, an MK2 inhibiting agent can be an sgRNA targeting MK2 or MK2R1.

Methods for preparing an MK2 inhibiting agent (e.g., an agent capable of inhibiting MK2 signaling) can comprise the construction of a protein/Ab scaffold containing the natural MK2 receptor as an MK2 neutralizing agent; developing inhibitors of the MK2 receptor “down-stream”; or developing inhibitors of the MK2 production “up-stream”.

Inhibiting MK2 can be performed by genetically modifying MK2 in a subject or genetically modifying a subject to reduce or prevent the expression of the MK2 gene, such as through the use of CRISPR-Cas9 or analogous technologies, wherein, such modification reduces or prevents MK2 signaling.

In some aspects, the MK2 inhibiting agent is a compound selected from ATI-450, PF-3644022 (CAS NO. 1276121-88-0), and CMPD-1 (CAS NO. 41179-33-3). Descriptions and representative chemical structures of the MK2 inhibiting agents ATI-450, PF-3644022, and CMPD-1 are provided below.

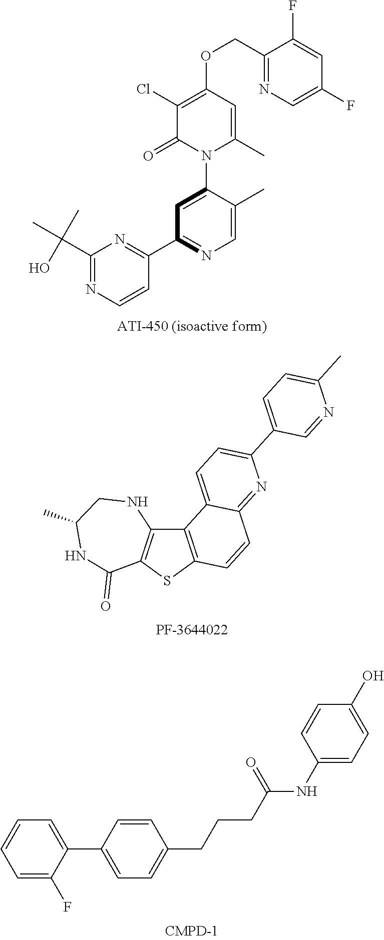

### Formulation

The agents and compositions described herein can be formulated in any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to modulate the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

In various aspects, a method of treating, preventing, or reversing pancreatic cancer in a subject in need is disclosed. The disclosed method includes the administration of a therapeutically effective amount of an MK2 inhibiting agent, so as to inhibit MK2 activation and/or downstream signaling. In some aspects, the method further includes the administration of a therapeutically effective amount of at least one chemotherapy compound. As demonstrated in the examples below, inhibition of the MK2 pathway by the MK2 inhibiting agent potentiates the efficacy of at least some chemotherapy compounds by overcoming or preventing the development of chemotherapeutic drug tolerance.

In various other aspects, a method of treating preventing, or reversing pancreatic cancer in a subject in need, wherein the subject in need exhibits resistance to chemotherapeutic treatment is disclosed that includes the administration of a therapeutically effective amount of an MK2 inhibiting agent and a therapeutically effective amount of at least one chemotherapy compound. As demonstrated in the examples below, the inhibition of MK2 activation and/or downstream signaling resulting from the administration of the MK2 inhibiting agent inhibits and/or reverses various biochemical pathways thought to be involved in the development of resistance to chemotherapeutic treatment.

In various additional aspects, a method of preventing, inhibiting, and/or reversing the development of chemotherapeutic resistance is disclosed that includes the administration of a therapeutically effective amount of an MK2 inhibiting agent. In some aspects, the MK2 inhibiting agent is administered along with a therapeutically effective amount of at least one chemotherapy compound. In other aspects, the MK2 inhibiting agent is administered to a patient already receiving a chemotherapeutic treatment to inhibit the further development of chemotherapeutic resistance and/or to reverse at least a portion of any chemotherapeutic resistance that has already developed.

Methods described herein are generally performed on a subject in need thereof. A subject in need of the therapeutic methods described herein can be a subject having, diagnosed with, suspected of having, or at risk for developing pancreatic cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of MK2 inhibitor is, for example, an amount that would cause the desired therapeutic effect in a subject while minimizing undesired side effects. In various embodiments, an effective amount of MK2 inhibitor described herein can substantially inhibit pancreatic cancer, slow the progress of pancreatic cancer, or limit the development of pancreatic cancer.

According to the methods described herein, the administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of MK2 inhibitor can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat or slow the progress of pancreatic cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of MK2 inhibitor can occur as a single event or over a time course of treatment. For example, the MK2 inhibitor can be administered daily, weekly, bi-weekly, or monthly. For the treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for pancreatic cancer.

An MK2 inhibitor can be administered simultaneously or sequentially with another agent, such as an antibiotic, an anti-inflammatory, a chemotherapy composition, or another agent. For example, an MK2 inhibitor can be administered simultaneously with one or more chemotherapy compounds. Simultaneous administration can occur through the administration of separate compositions, each containing one or more of an MK2 inhibitor, one or more chemotherapy compounds, an antibiotic, an anti-inflammatory, or another agent. Simultaneous administration can occur through the administration of one composition containing two or more of an MK2 inhibitor, one or more chemotherapy compounds, an antibiotic, an anti-inflammatory, or another agent. An MK2 inhibitor can be administered sequentially with one or more chemotherapy compounds, an antibiotic, an anti-inflammatory, or another agent. For example, an MK2 inhibitor can be administered before or after administration of one or more chemotherapy compounds, an antibiotic, an anti-inflammatory, or another agent.

Non-limiting examples of suitable chemotherapy compounds to be co-administered with the MK2 inhibitor include Abiraterone Acetate; Abitrexate (Methotrexate); Abraxane (Paclitaxel Albumin-stabilized Nanoparticle Formulation); ABVD; ABVE; ABVE-PC; AC; AC-T; Adcetris (Brentuximab Vedotin); ADE; Ado-Trastuzumab Emtansine; Adriamycin (Doxorubicin Hydrochloride); Afatinib Dimaleate; Afinitor (Everolimus); Akynzeo (Netupitant and Palonosetron Hydrochloride); Aldara (Imiquimod); Aldesleukin; Alecensa (Alectinib); Alectinib; Alemtuzumab; Alkeran (Melphalan Hydrochloride); Alkeran (Melphalan); Alimta (Pemetrexed Disodium); Aloxi (Palonosetron Hydrochloride); Ambochlorin/Amboclorin (Chlorambucil); Amifostine; Aminolevulinic Acid; Anastrozole; Aprepitant; Aredia (Pamidronate Disodium); Arimidex (Anastrozole); Aromasin (Exemestane); Arranon (Nelarabine); Arsenic Trioxide; Arzerra (Ofatumumab); Asparaginase Erwinia chrysanthemi; Atezolizumab; Avastin (Bevacizumab); Avelumab; Axitinib; Azacitidine; Bavencio (Avelumab); BEACOPP; Becenum (Carmustine); Beleodaq (Belinostat); Belinostat; Bendamustine Hydrochloride; BEP; Bevacizumab; Bexarotene; Bexxar (Tositumomab and Iodine I 131 Tositumomab); Bicalutamide; BiCNU (Carmustine); Bleomycin; Blinatumomab; Blincyto (Blinatumomab); Bortezomib; Bosulif (Bosutinib); Bosutinib; Brentuximab Vedotin; BuMel; Busulfan; Busulfex (Busulfan); Cabazitaxel; Cabometyx (Cabozantinib-S-Malate); Cabozantinib-S-Malate; CAF; Campath (Alemtuzumab); Camptosar (Irinotecan Hydrochloride); Capecitabine; CAPDX; Carac (Fluorouracil—Topical); Carboplatin; Carboplatin-Taxol; Carfilzomib; Carmubris (Carmustine); Casodex (Bicalutamide); CEM; Ceritinib; Cerubidine (Daunorubicin Hydrochloride); Cervarix (Recombinant HPV Bivalent Vaccine); Cetuximab; CEV; Chlorambucil; Chlorambucil-prednisone; CHOP; Cisplatin; Cladribine; Clafen (Cyclophosphamide); Clofarabine; Clofarex (Clofarabine); Clolar (Clofarabine); CMF; Cobimetinib; Cometriq (Cabozantinib-S-Malate); COPDAC; COPP; COPP-ABV; Cosmegen (Dactinomycin); Cotellic (Cobimetinib); Crizotinib; CVP; Cyclophosphamide; Cyfos (Ifosfamide); Cyramza (Ramucirumab); Cytarabine; Cytarabine Liposome; Cytosar-U (Cytarabine); Cytoxan (Cyclophosphamide); Dabrafenib; Dacarbazine; Dacogen (Decitabine); Dactinomycin; Daratumumab; Darzalex (Daratumumab); Dasatinib; Daunorubicin Hydrochloride; Decitabine; Defibrotide Sodium; Defitelio (Defibrotide Sodium); Degarelix; Denileukin Diftitox; Denosumab; DepoCyt (Cytarabine Liposome); Dexamethasone; Dexrazoxane Hydrochloride; Dinutuximab; Docetaxel; Doxil (Doxorubicin Hydrochloride Liposome); Doxorubicin Hydrochloride; Doxorubicin Hydrochloride Liposome; Dox-SL (Doxorubicin Hydrochloride Liposome); DTIC-Dome (Dacarbazine); Efudex (Fluorouracil—Topical); Elitek (Rasburicase); Ellence (Epirubicin Hydrochloride); Elotuzumab; Eloxatin (Oxaliplatin); Eltrombopag Olamine; Emend (Aprepitant); Empliciti (Elotuzumab); Enzalutamide; Epirubicin Hydrochloride; EPOCH; Erbitux (Cetuximab); Eribulin Mesylate; Erivedge (Vismodegib); Erlotinib Hydrochloride; Erwinaze (Asparaginase Erwinia chrysanthemi); Ethyol (Amifostine); Etopophos (Etoposide Phosphate); Etoposide; Etoposide Phosphate; Evacet (Doxorubicin Hydrochloride Liposome); Everolimus; Evista (Raloxifene Hydrochloride); Evomela (Melphalan Hydrochloride); Exemestane; 5-FU (Fluorouracil Injection); 5-FU (Fluorouracil—Topical); Fareston (Toremifene); Farydak (Panobinostat); Faslodex (Fulvestrant); FEC; Femara (Letrozole); Filgrastim; Fludara (Fludarabine Phosphate); Fludarabine Phosphate; Fluoroplex (Fluorouracil—Topical); Fluorouracil Injection; Fluorouracil—Topical; Flutamide; Folex (Methotrexate); Folex PFS (Methotrexate); FOLFIRI; FOLFIRI-BEVACIZUMAB; FOLFIRI-CETUXIMAB; FOLFIRINOX; FOLFOX; Folotyn (Pralatrexate); FU-LV; Fulvestrant; Gardasil (Recombinant HPV Quadrivalent Vaccine); Gardasil 9 (Recombinant HPV Nonavalent Vaccine); Gazyva (Obinutuzumab); Gefitinib; Gemcitabine Hydrochloride; Gemcitabine-Cisplatin; GEMCITABINE-OXALIPLATIN; Gemtuzumab Ozogamicin; Gemzar (Gemcitabine Hydrochloride); Gilotrif (Afatinib Dimaleate); Gleevec (Imatinib Mesylate); Gliadel (Carmustine Implant); Gliadel wafer (Carmustine Implant); Glucarpidase; Goserelin Acetate; Halaven (Eribulin Mesylate); Hemangeol (Propranolol Hydrochloride); Herceptin (Trastuzumab); HPV Bivalent Vaccine, Recombinant; HPV Nonavalent Vaccine, Recombinant; HPV Quadrivalent Vaccine, Recombinant; Hycamtin (Topotecan Hydrochloride); Hydrea (Hydroxyurea); Hydroxyurea; Hyper-CVAD; Ibrance (Palbociclib); Ibritumomab Tiuxetan; Ibrutinib; ICE; Iclusig (Ponatinib Hydrochloride); Idamycin (Idarubicin Hydrochloride); Idarubicin Hydrochloride; Idelalisib; Ifex (Ifosfamide); Ifosfamide; Ifosfamidum (Ifosfamide); IL-2 (Aldesleukin); Imatinib Mesylate; Imbruvica (Ibrutinib); Imiquimod; Imlygic (Talimogene Laherparepvec); Inlyta (Axitinib); Interferon Alfa-2b, Recombinant; Interleukin-2 (Aldesleukin); Intron A (Recombinant Interferon Alfa-2b); Iodine I 131 Tositumomab and Tositumomab; Ipilimumab; Iressa (Gefitinib); Irinotecan Hydrochloride; Irinotecan Hydrochloride Liposome; Istodax (Romidepsin); Ixabepilone; Ixazomib Citrate; Ixempra (Ixabepilone); Jakafi (Ruxolitinib Phosphate); JEB; Jevtana (Cabazitaxel); Kadcyla (Ado-Trastuzumab Emtansine); Keoxifene (Raloxifene Hydrochloride); Kepivance (Palifermin); Keytruda (Pembrolizumab); Kisqali (Ribociclib); Kyprolis (Carfilzomib); Lanreotide Acetate; Lapatinib Ditosylate; Lartruvo (Olaratumab); Lenalidomide; Lenvatinib Mesylate; Lenvima (Lenvatinib Mesylate); Letrozole; Leucovorin Calcium; Leukeran (Chlorambucil); Leuprolide Acetate; Leustatin (Cladribine); Levulan (Aminolevulinic Acid); Linfolizin (Chlorambucil); LipoDox (Doxorubicin Hydrochloride Liposome); Lomustine; Lonsurf (Trifluridine and Tipiracil Hydrochloride); Lupron (Leuprolide Acetate); Lupron Depot (Leuprolide Acetate); Lupron Depot-Ped (Leuprolide Acetate); Lynparza (Olaparib); Marqibo (Vincristine Sulfate Liposome); Matulane (Procarbazine Hydrochloride); Mechlorethamine Hydrochloride; Megestrol Acetate; Mekinist (Trametinib); Melphalan; Melphalan Hydrochloride; Mercaptopurine; Mesna; Mesnex (Mesna); Methazolastone (Temozolomide); Methotrexate; Methotrexate LPF (Methotrexate); Methylnaltrexone Bromide; Mexate (Methotrexate); Mexate-AQ (Methotrexate); Mitomycin C; Mitoxantrone Hydrochloride; Mitozytrex (Mitomycin C); MOPP; Mozobil (Plerixafor); Mustargen (Mechlorethamine Hydrochloride); Mutamycin (Mitomycin C); Myleran (Busulfan); Mylosar (Azacitidine); Mylotarg (Gemtuzumab Ozogamicin); Nanoparticle Paclitaxel (Paclitaxel Albumin-stabilized Nanoparticle Formulation); Navelbine (Vinorelbine Tartrate); Necitumumab; Nelarabine; Neosar (Cyclophosphamide); Netupitant and Palonosetron Hydrochloride; Neulasta (Pegfilgrastim); Neupogen (Filgrastim); Nexavar (Sorafenib Tosylate); Nilandron (Nilutamide); Nilotinib; Nilutamide; Ninlaro (Ixazomib Citrate); Nivolumab; Nolvadex (Tamoxifen Citrate); Nplate (Romiplostim); Obinutuzumab; Odomzo (Sonidegib); OEPA; Ofatumumab; OFF; Olaparib; Olaratumab; Omacetaxine Mepesuccinate; Oncaspar (Pegaspargase); Ondansetron Hydrochloride; Onivyde (Irinotecan Hydrochloride Liposome); Ontak (Denileukin Diftitox); Opdivo (Nivolumab); OPPA; Osimertinib; Oxaliplatin; Paclitaxel; Paclitaxel Albumin-stabilized Nanoparticle Formulation; PAD; Palbociclib; Palifermin; Palonosetron Hydrochloride; Palonosetron Hydrochloride and Netupitant; Pamidronate Disodium; Panitumumab; Panobinostat; Paraplat (Carboplatin); Paraplatin (Carboplatin); Pazopanib Hydrochloride; PCV; PEB; Pegaspargase; Pegfilgrastim; Peginterferon Alfa-2b; PEG-Intron (Peginterferon Alfa-2b); Pembrolizumab; Pemetrexed Disodium; Perjeta (Pertuzumab); Pertuzumab; Platinol (Cisplatin); Platinol-AQ (Cisplatin); Plerixafor; Pomalidomide; Pomalyst (Pomalidomide); Ponatinib Hydrochloride; Portrazza (Necitumumab); Pralatrexate; Prednisone; Procarbazine Hydrochloride; Proleukin (Aldesleukin); Prolia (Denosumab); Promacta (Eltrombopag Olamine); Propranolol Hydrochloride; Provenge (Sipuleucel-T); Purinethol (Mercaptopurine); Purixan (Mercaptopurine); Radium 223 Dichloride; Raloxifene Hydrochloride; Ramucirumab; Rasburicase; R-CHOP; R-CVP; Recombinant Human Papillomavirus (HPV) Bivalent Vaccine; Recombinant Human Papillomavirus (HPV) Nonavalent Vaccine; Recombinant Human Papillomavirus (HPV) Quadrivalent Vaccine; Recombinant Interferon Alfa-2b; Regorafenib; Relistor (Methylnaltrexone Bromide); R-EPOCH; Revlimid (Lenalidomide); Rheumatrex (Methotrexate); Ribociclib; R-ICE; Rituxan (Rituximab); Rituximab; Rolapitant Hydrochloride; Romidepsin; Romiplostim; Rubidomycin (Daunorubicin Hydrochloride); Rubraca (Rucaparib Camsylate); Rucaparib Camsylate; Ruxolitinib Phosphate; Sclerosol Intrapleural Aerosol (Talc); Siltuximab; Sipuleucel-T; Somatuline Depot (Lanreotide Acetate); Sonidegib; Sorafenib Tosylate; Sprycel (Dasatinib); STANFORD V; Sterile Talc Powder (Talc); Steritalc (Talc); Stivarga (Regorafenib); Sunitinib Malate; Sutent (Sunitinib Malate); Sylatron (Peginterferon Alfa-2b); Sylvant (Siltuximab); Synribo (Omacetaxine Mepesuccinate); Tabloid (Thioguanine); TAC; Tafinlar (Dabrafenib); Tagrisso (Osimertinib); Talc; Talimogene Laherparepvec; Tamoxifen Citrate; Tarabine PFS (Cytarabine); Tarceva (Erlotinib Hydrochloride); Targretin (Bexarotene); Tasigna (Nilotinib); Taxol (Paclitaxel); Taxotere (Docetaxel); Tecentriq (Atezolizumab); Temodar (Temozolomide); Temozolomide; Temsirolimus; Thalidomide; Thalomid (Thalidomide); Thioguanine; Thiotepa; Tolak (Fluorouracil—Topical); Topotecan Hydrochloride; Toremifene; Torisel (Temsirolimus); Tositumomab and Iodine I 131 Tositumomab; Totect (Dexrazoxane Hydrochloride); TPF; Trabectedin; Trametinib; Trastuzumab; Treanda (Bendamustine Hydrochloride); Trifluridine and Tipiracil Hydrochloride; Trisenox (Arsenic Trioxide); Tykerb (Lapatinib Ditosylate); Unituxin (Dinutuximab); Uridine Triacetate; VAC; Vandetanib; VAMP; Varubi (Rolapitant Hydrochloride); Vectibix (Panitumumab); VeIP; Velban (Vinblastine Sulfate); Velcade (Bortezomib); Velsar (Vinblastine Sulfate); Vemurafenib; Venclexta (Venetoclax); Venetoclax; Viadur (Leuprolide Acetate); Vidaza (Azacitidine); Vinblastine Sulfate; Vincasar PFS (Vincristine Sulfate); Vincristine Sulfate; Vincristine Sulfate Liposome; Vinorelbine Tartrate; VIP; Vismodegib; Vistogard (Uridine Triacetate); Voraxaze (Glucarpidase); Vorinostat; Votrient (Pazopanib Hydrochloride); Wellcovorin (Leucovorin Calcium); Xalkori (Crizotinib); Xeloda (Capecitabine); XELIRI; XELOX; Xgeva (Denosumab); Xofigo (Radium 223 Dichloride); Xtandi (Enzalutamide); Yervoy (Ipilimumab); Yondelis (Trabectedin); Zaltrap (Ziv-Aflibercept); Zarxio (Filgrastim); Zelboraf (Vemurafenib); Zevalin (Ibritumomab Tiuxetan); Zinecard (Dexrazoxane Hydrochloride); Ziv-Aflibercept; Zofran (Ondansetron Hydrochloride); Zoladex (Goserelin Acetate); Zoledronic Acid; Zolinza (Vorinostat); Zometa (Zoledronic Acid); Zydelig (Idelalisib); Zykadia (Ceritinib); or Zytiga (Abiraterone Acetate). In some aspects, the MK2 inhibitor is co-adminstered with a irinotecan-containing chemotherapy composition including, but not limited to, FOLFIRINOX (a cocktail of folinic acid, 5-FU, irinotecan, and oxaliplatin), FOLFOX (a cocktail of leucovorin calcium (folinic acid), fluorouracil, and oxaliplatin), FOLFOX/CD40 agonist/anti-PD1 cocktail, FOLFOX/anti-CTLA4/anti-PD1 cocktail, and gemcitabine/nab-paclitaxel.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, the administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump that may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow co-localized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to MK2 inhibitors and one or more chemotherapy compounds. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following examples illustrate various aspects of the disclosure.

### Example 1: Proteomics Reveal MK2/Hsp27 Axis as a Major Survival Mechanism Following Genotoxic Stress

Combination chemotherapies remain the cornerstone treatment for patients with gastrointestinal malignancies. Pancreatic ductal adenocarcinoma (PDAC) is perhaps the most treatment refractory, as de novo or acquired resistance is near-universal. The following experiments were conducted to identify and characterize resistance mechanisms to cytotoxic chemotherapy in PDAC.

For these experiments, the FOLFIRINOX regimen was employed, as it is the most aggressive regimen currently used in practice for patients with advanced/metastatic pancreatic cancer. Using an unbiased reverse-phase protein array, activation of heat-shock protein 27 (Hsp27) was identified as the most significantly upregulated event following FOLFIRINOX treatment in PDAC cells. Silencing of Hsp27 by RNA interference (RNAi), or by suppression of its upstream kinase MAPKAPK2 (or MK2) by a small molecule inhibitor or an RNAi, significantly augmented the apoptotic effect of FOLFIRINOX.

Mechanistically, SN38 (the active metabolite of irinotecan) dramatically upregulated TNFα, leading to TNFα-dependent autocrine phosphorylation of TAK1, MK2, and Hsp27. Targeting MK2 abrogated Hsp27 activation, sensitized cells to apoptosis, and additionally suppressed SN38-induced protective autophagy, in part by suppressing phosphorylation of Beclin-1.

In an autochthonous PDAC mouse model, the novel MK2 inhibitor ATI-450 significantly retarded PDAC development and progression, which when combined with FOLFIRINOX almost completely ablated all PDAC foci and significantly improved survival. Consistent with preclinical data, high phospho-MK2 was associated with significantly inferior survival in PDAC subjects. Overall, the results of these experiments identified MK2 as a critical mediator of genotoxic stress-induced activation of pro-survival pathways and provided preclinical justification for combining an MK2 inhibitor with irinotecan-containing chemotherapies in various solid tumors.

### Methods

In general, all experiments were replicated two to four times. Critical observations were replicated with multiple cell lines. Animal measurements and analysis were conducted in a blinded manner using animal numbers to prevent bias.

### Cell Culture

Cells were cultured in Dulbecco's Modified Eagle's Medium (DMEM) with 10% Fetal Bovine Serum (FBS), 1% penicillin/streptomycin/amphotericin B, and cultured at 37 C in 5% CO2. Mycoplasma testing was performed yearly using the MycoSEQ Detection kit (Applied Biosystems). All lines were used for fewer than 6 months after receipt or thawing

### Drugs and Reagents

Gemcitabine was purchased from a clinical pharmacy. 5-fluorouracil (5-FU) and oxaliplatin were obtained from Sigma. SN38 and PF-36344022 were purchased from Selleckchem. ATI-450 was provided by Aclaris Therapeutics LLC. All other agents were purchased: (5Z)-7-Oxozeaenol (Tocris, cat #3604), hydroxychloroquine (5648/10), TNFα (BioLegend, cat #570104), Trametinib (Selleckchem, cat #S2673), AS2444697 (Tocris, cat #5430), KU55933 (Selleckchem, cat #S1092), VE821 (Selleckchem, cat #S8007), and SP600125 (Selleckchem, cat #S1460), IMD0354 (Tocris, cat #2611), GSK963 (Selleckchem cat #S8642).

### Tumor Sectioning, IHC, and Immunofluorescence

Tumors were formalin-fixed, paraffin-embedded, sectioned, and stained with hematoxylin and eosin (H&E). Immunohistochemistry (IHC) staining was performed using the following antibodies: p-Hsp27 S82 (CST cat #9709S), p-MK2 T334 (CST cat #3041S), p-p38 T180/Y182 (CST cat #9215S). Ten 200× or 400× power fields were analyzed per tumor independently by two individuals, and data were presented as mean+SEM of collated data. IHC sections were interpreted independently by two individual reviewers and representative data agreeable to all members were presented.

### Soft Agar Assay

20,000 cells were suspended in 0.6% Noble agar and seeded in triplicate in 24-well plates. Wells were re-treated weekly to prevent desiccation. After 3 to 4 weeks of growth, colony numbers from each well were counted manually using a dissecting microscope.

### Reverse Phase Protein Array (RPPA)

Pa01c cells were treated with FIRINOX (5-FU 10 μM, SN38 10 μM, oxaliplatin 10 μM) for 16 hours, washed twice in ice-cold 1×PBS, and lysates were prepared according to well-established guidelines. Normalized linear values were log 2 transformed, and median centered for analysis.

### Immunoblotting

Western blotting was performed according to standard procedures. Cells grown in culture were washed twice in ice-cold 1×PBS and lysed in ice-cold RIPA buffer (10 nM Tris pH 7.4, 50 nM NaCl, 0.05% SDS, 0.05% Triton X-100, 0.5% sodium deoxycholate, and 10 nM EDTA) containing 1× protease inhibitor (Sigma), NaVO3, Na pyrophosphate, and NaF. Protein lysates were quantified by Bradford assay and suspended in 6×SDS sample buffer, followed by boiling for 5 min at 95 C. 40 ug of protein lysate was resolved on SDS-PAGE and blotted on nitrocellulose membrane. Antibodies used included p38 MAPK (CST 8690S), p38 MAPK T180/Y182 (CST 9215S), MK2 total (CST 12155S), MK2 T334 (CST 3041S), MK2 T222 (3316S), Hsp27 total (CST 2042S), Hsp27 phos S82 (9709S), [symbol] H2Ax (CST 9718S), JNK T183/Y185 (CST 4668), GSK3 beta phos S9 (CST 5558), ATR phos T1989 (CST 58014), ATM phos 51981 (CST 4526), AMPKα1 phos T172 (CST 2335T), and GAPDH (sc-47724). Blots were imaged using chemiluminescent substrate (Thermo Scientific).

### RNA Interference

Short hairpin RNA (shRNA) was used to silence gene expression of MK2, Hsp27, and TNF receptor (TNFR) by RNA interference (RNAi) using the target sequences and shRNA sequences summarized in Tables 1 and 2 below:

TABLE 1
shRNA Target Sequences for RNAi

| SEQ | | | |
|---|---|---|---|
| ID | | | Target ID Seq |
| NO | Target | Sequence | Number |
| 1 | MK2-1 | GACTACGAGCAGATCAAGATA | TRCN0000002283 |
| 2 | MK2-2 | AGAAAGAGAAGCATCCGAAAT | TRCN0000002286 |

TABLE 2
shRNA Sequences for RNAi

| SEQ | | |
|---|---|---|
| ID | | |
| NO | shRNA | Sequence |
| 3 | Hsp27-1 | CCGGCCCGGACGAGCTGACGGTCAACTCGAGTTGA |
| | (fwd) | CCGTCAGCTCGTCCGGGTTTTTG |
| 4 | Hsp27-1 | AATTCAAAAACCCGGACGAGCTGACGGTCAACTC |
| | (rev) | GAGTTGACCGTCAGCTCGTCCGGG |
| 5 | Hsp27-2 | CCGGGATCACCATCCCAGTCACCTTCTCGAGAAG |
| | (fwd) | GTGACTGGGATGGTGATCTTTTTG |
| 6 | Hsp27-2 | AATTCAAAAAGATCACCATCCCAGTCACCTTCTCG |
| | (rev) | AGAAGGTGACTGGGATGGTGATC |
| 7 | TNFR | CCGGGGAGCTGTTGGTGGGAATATACTCGAGTAT |
| | (fwd) | ATTCCCACCAACAGCTCCTTTTTG |
| 8 | TNFR | AATTCAAAAAGGAGCTGTTGGTGGGAATATACTC |
| | (rev) | GAGTATATTCCCACCAACAGCTCC |

### Quantitative (Real-Time) PCR

RNAzol RT (Sigma) was used to isolate total RNA. cDNA was made with High Capacity cDNA reverse transcription kit (Thermo Fisher Scientific, 4368814). qPCR was performed with SYBR-Green reagent (Applied Biosciences). All experiments were performed in biological duplicates or triplicates (n=2, 3). Primer sequences are summarized in Table 3 below:

TABLE 3
Primer Sequences for rtPCR

| SEQ | | |
|---|---|---|
| ID | | |
| NO | Primer | Sequence |
| 9 | TNFα (fwd) | GGAGAAGGGTGACCGACTCA |
| 10 | TNFα (rev) | CTGCCCAGACTCGGCAA |
| 11 | TNFα (fwd) | TACACCTCCTCCTTCTGGGG |
| 12 | TNFα (rev) | TCCAATGAGGTGAGCAGCAG |
| 13 | IL1α (fwd) | CACCTTTTAGCTTCCTGAC |
| 14 | IL1α (rev) | AATTTCACTGCTTCATCCAG |
| 15 | IL1β (fwd) | CTAAACAGATGAAGTGCTCC |
| 16 | IL1β (rev) | GGTCATTCTCCTGGAAGG |
| 17 | TGFβ (fwd) | CCCACAACGAAATCTATGAC |
| 18 | TGFβ (rev) | TGTATTTCTGGTACAGCTCC |

### Xenograft Tumorigenesis Assay

Human pancreatic cancer cell lines MIA Paca-2 and Pa01c (2×10⁶) were suspended in MatriGel in PBS and injected subcutaneously into bilateral flanks of NU/J nude female mice. Treatment was initiated when tumors became palpable but were less than 200 mm³. Tumors were serially measured until mice were sacrificed when humane endpoints were reached.

### Autochthonous Mouse Model

KPPC (p48-Cre/p53Flox/Flox LSL-KRASG12D) mice were created as described in previous publications. Treatment began six weeks after birth when tumors spontaneously form in vivo. FOLFIFINOX treatment was given by weekly intraperitoneal (I.P.) injection of 5-FU 25 mg/kg, irinotecan 17.5 mg/kg, and oxaliplatin 3.3 mg/kg. ATI-450 treatment was given as drug-impregnated chow, ad libitum, starting six weeks after birth; when given in combination with chemotherapy, ATI-450 chow was begun the day prior to initiation of chemotherapy.

### Statistical Analysis

Statistical analyses were performed using the GraphPad Prism v7/8 software and when applicable are expressed as mean±SEM. Comparisons between the two groups were done via Unpaired student's two-tailed t-tests. One-way or two-way ANOVA with post-test was used for multiple groups. Repeated measures ANOVA was utilized for systemic/group variation. For all comparisons, P values <0.05 were considered statistically significant. Cox proportional hazards models were used to analyze clinical characteristics in relation to overall survival. SAS version 9.4 (SAS Institute, Cary, N.C.) was used to generate Kaplan-Meier curves and were analyzed by log-rank tests.

### Results

### FOLFIRINOX Activates Pro-Survival Hsp27 in PDAC Cells

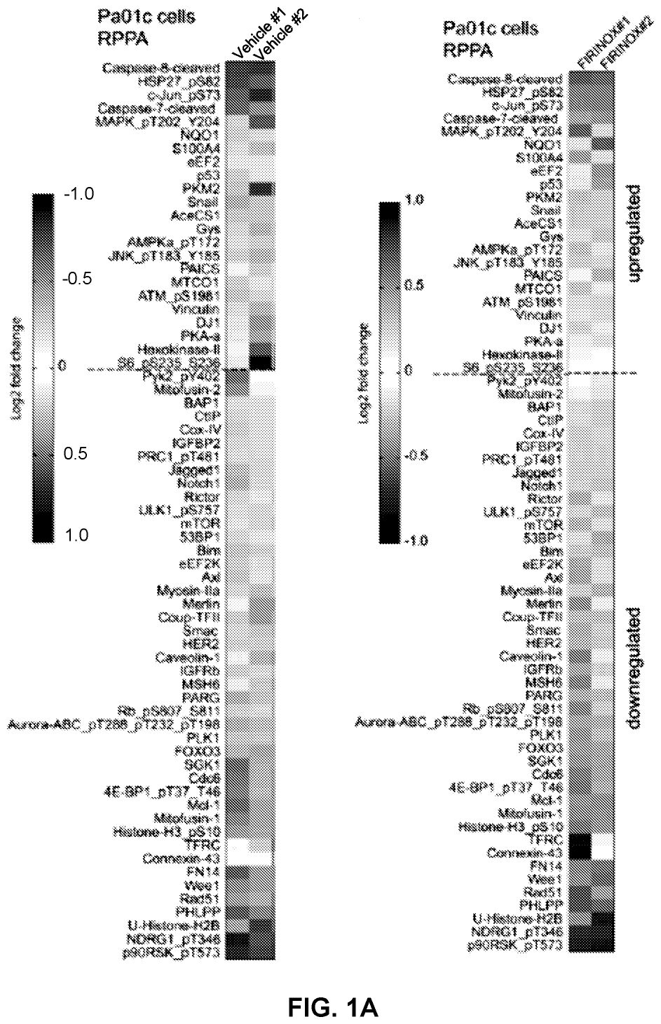

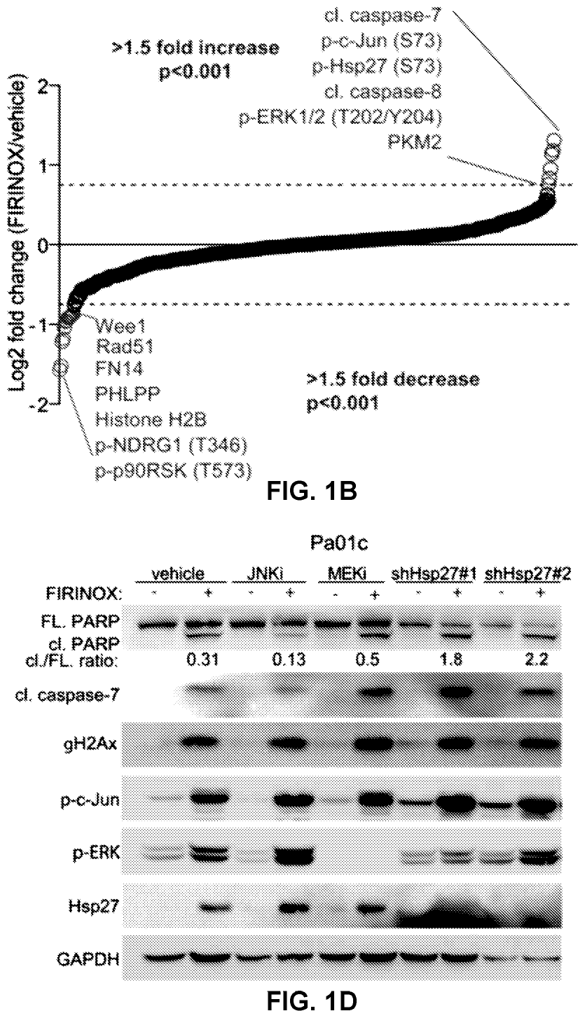

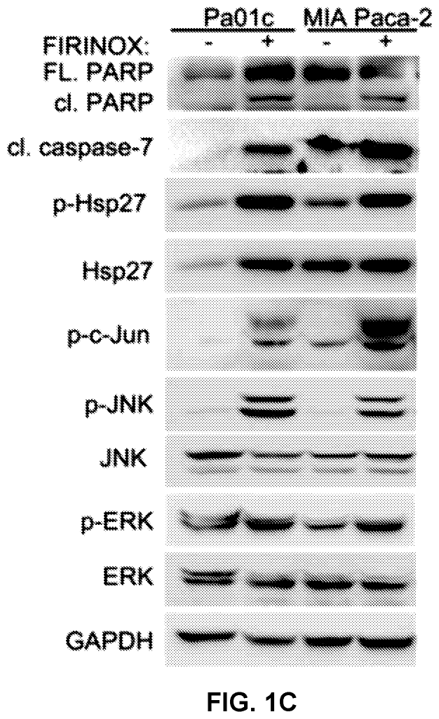

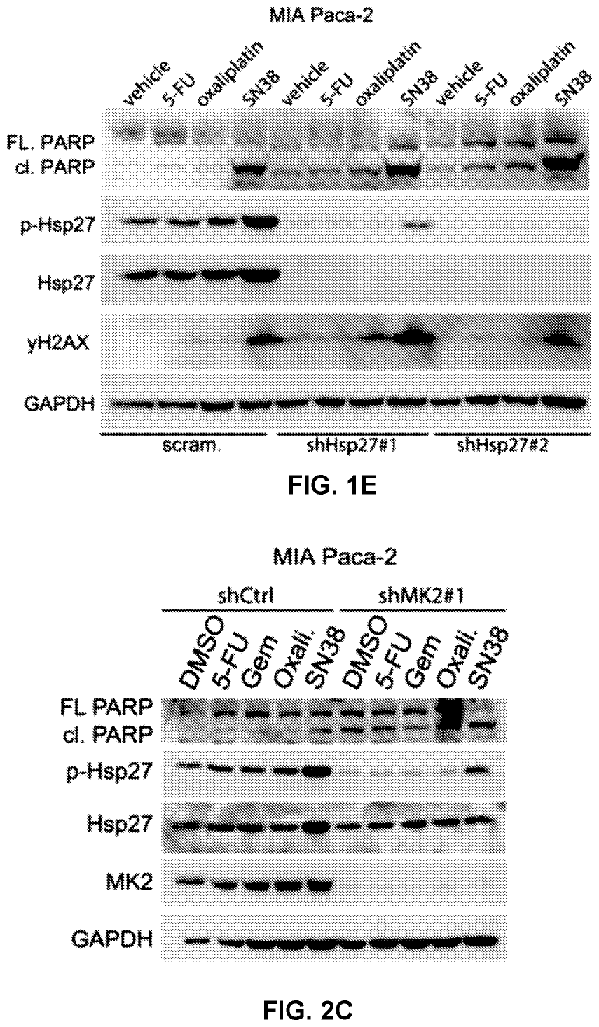

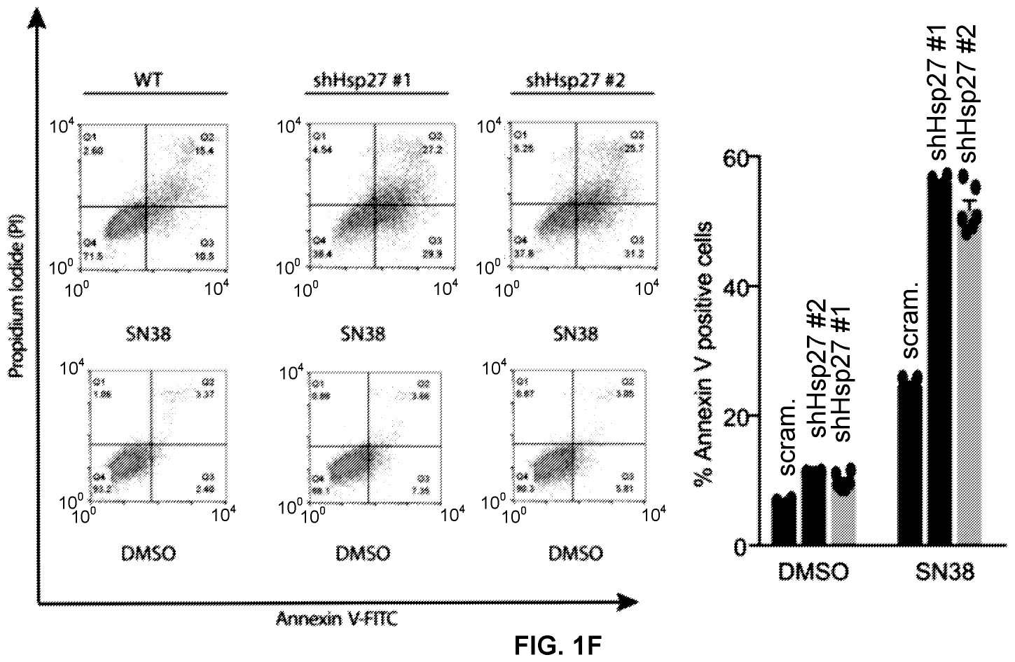

To comprehensively study the survival mechanisms following exposure to cytotoxic chemotherapy, an unbiased reverse-phase protein array (RPPA) was performed using Pa01c cells, a KRAS- and TP53-mutant PDAC PDCL, following exposure to a sub-lethal dose of FOLFIRINOX. Dosages of each agent in this cocktail were carefully selected so that >50% of Pa01c cells were killed after treatment for 96 hours, which mimics the clinical scenario. To capture both apoptotic and survival events, cells were treated for 16 hours and harvested for analysis. Of 441 markers tested, phosphorylated (p-) Hsp27 (S82), c-Jun and ERK1/2 were found to be the most significantly upregulated (defined as >1.5 fold) following FOLFIRINOX treatment (FIGS. 1A, 1B). Notably, significantly upregulated cleaved caspase-7 was also observed, indicating engagement of death mechanisms. These findings were confirmed in Pa01c and additionally MIA Paca-2 cells by western blots (FIG. 1C). On the other hand, Weel and Rad51 levels were significantly downregulated by FOLFIRINOX, implying extensive double-strand breaks and impairment of homologous recombination, although the mechanism is unclear.

To determine the role of upregulated p-Hsp27, p-c-Jun, and p-ERK1/2, Pa01c cells were treated with FOLFIRINOX alone or concurrently with a JNK inhibitor (SP600125), MEK inhibitor (trametinib), or with the expression of two small hairpin sequences that silence Hsp27 (shHsp27, Table 2). Notably, silencing of Hsp27 was the most effective in augmenting apoptosis (>6 fold), assayed by PARP cleavage and induction of cleaved caspase-7 (FIG. 1D). Trametinib modestly augmented, whereas JNKi intriguingly reversed, the apoptotic effect of FOLFIRINOX. Notably, suppression of MEK-ERK, JNK, or Hsp27 did not alter the severity of FOLFIRINOX-induced DNA damage, as assayed by [symbol] H2Ax levels, suggesting that the increase in apoptosis is due to a decreased survival threshold. Similarly, as assessed by FACS analysis, the addition of JNKi or MEKi to FOLFIRINOX did not appreciably augment apoptosis.

To delineate the chemotherapeutic agent in the FOLFIRINOX cocktail that most potently activates p-Hsp27, MIA Paca-2 cells were treated with vehicle, 5-FU, SN38, or oxaliplatin individually, and SN38 was identified as the agent that most strongly activates Hsp27 (FIG. 1F). For each agent, upregulation of p-Hsp27 positively correlated with [symbol] H2Ax levels, suggesting Hsp27 activation as a response to the extent of DNA damage, rather than related to the mechanism of action of each chemotherapeutic agent. Similarly, silencing of Hsp27 significantly promoted the apoptotic effect of SN38 (FIGS. 1F, 1G). These data demonstrated Hsp27 activation to be a pro-survival mechanism following FOLFIRINOX treatment in PDAC.

### FOLFIRINOX Activates Hsp27 Through MK2

Hsp27 is a molecular chaperone that has an anti-apoptotic function via binding and preventing cytochrome c from triggering the apoptosome. The binding of Hsp27 to its client proteins is regulated by phosphorylation. Phosphorylation of Hsp27, especially at S78 and S82, promotes the formation of small Hsp27 oligomers which enhances its chaperone function. Several stress kinases, particularly AKT and MAPK Activated Protein Kinase 2 (MAPKAPK2, or MK2), have been shown to be the major activators of Hsp27. From the RPPA, no significant change in p-AKT (S473 and T308) levels were observed following FOLFIRINOX treatment, prompting a focus on p-MK2, which was not included in the RPPA panel.

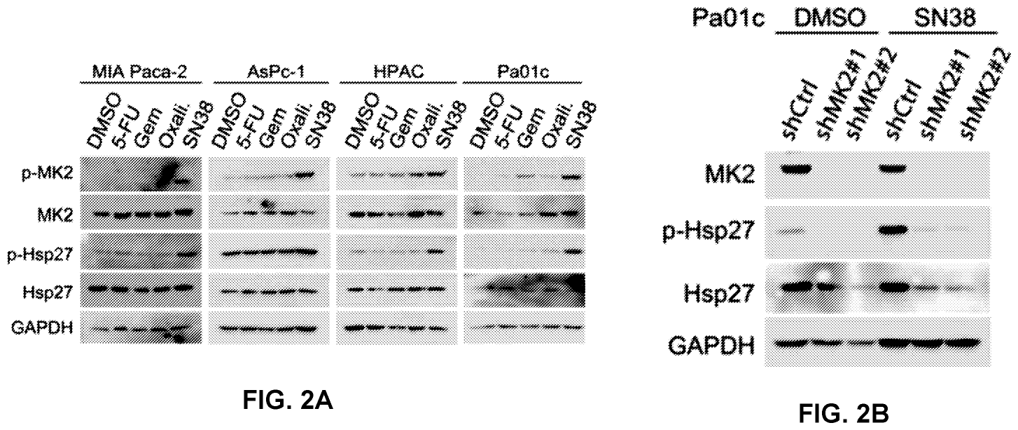

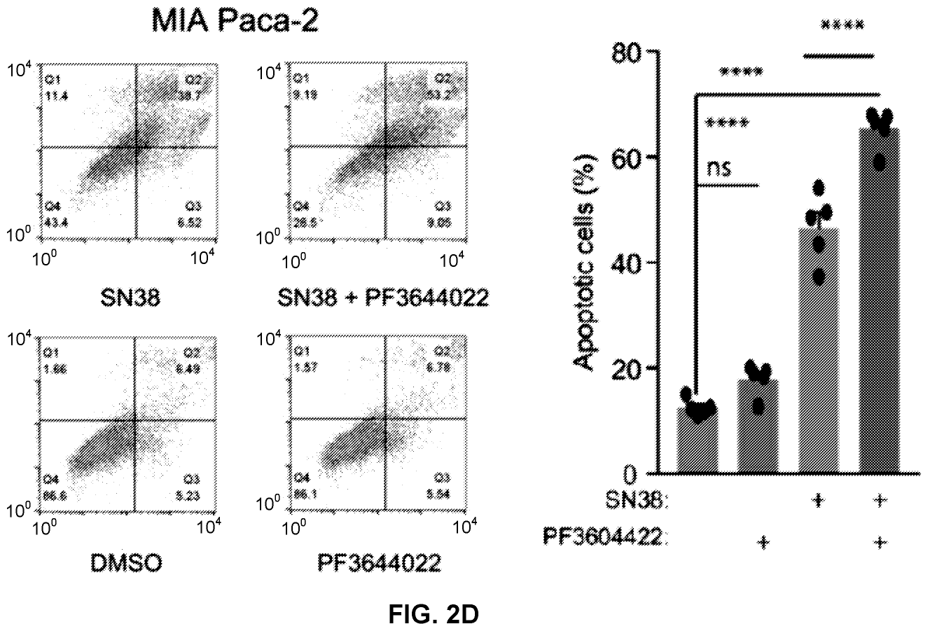

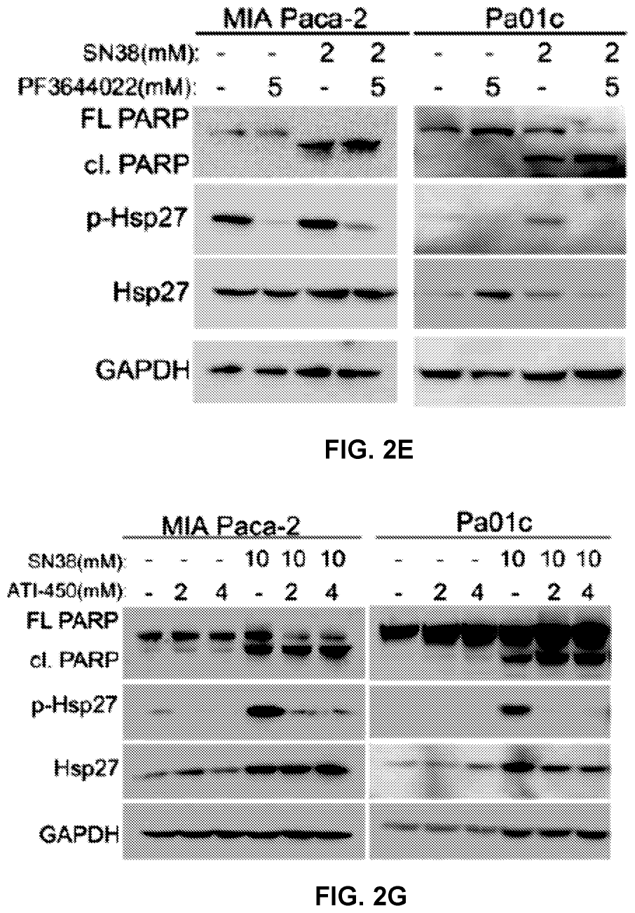

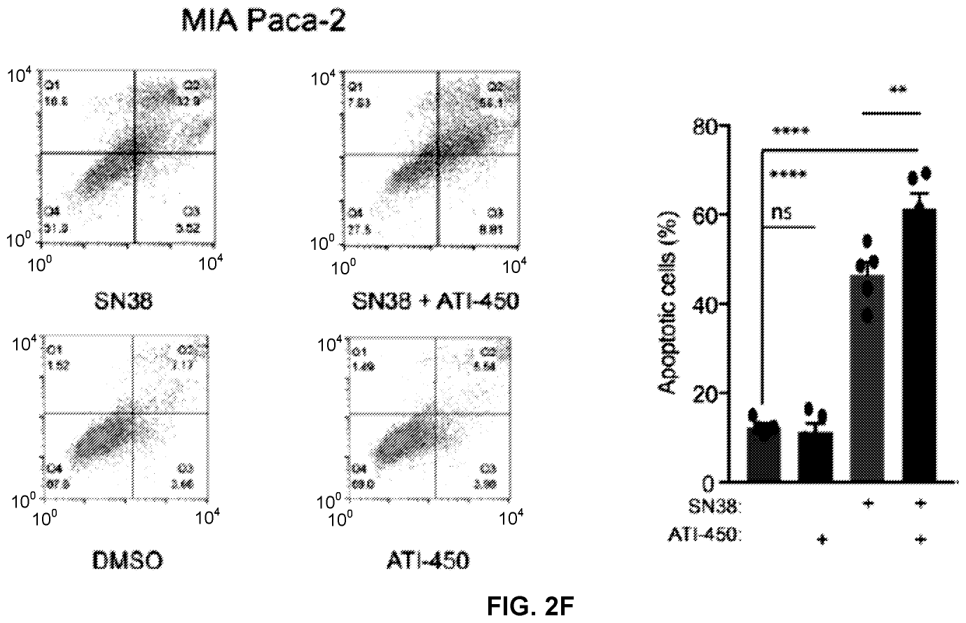

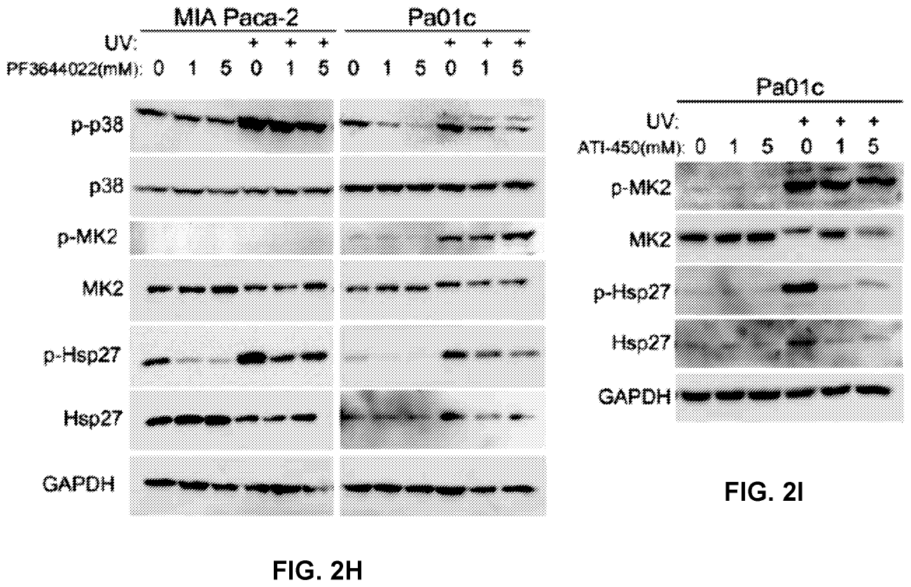

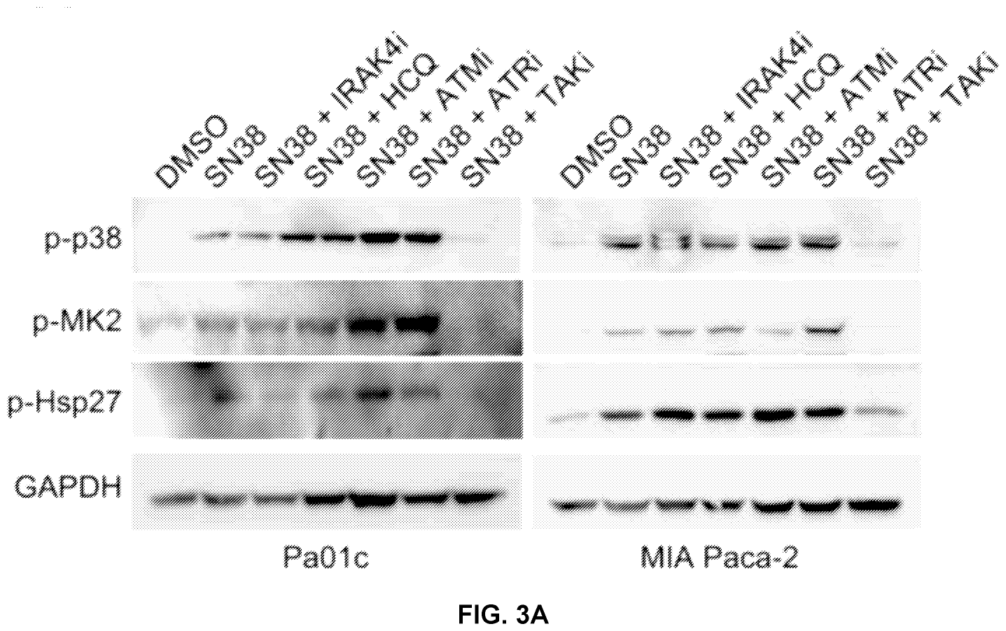

In all four PDAC lines we tested, SN38 potently induced both p-MK2 and p-Hsp27, implying MK2 as the upstream kinase for Hsp27 (FIG. 2A). Silencing of MK2 abolished p-Hsp27 at baseline and completely blocked SN38-induced upregulation of p-Hsp27 (FIG. 2B), leading to increased apoptosis assayed by PARP cleavage (FIG. 2C). Silencing of MK2 did not affect SN38-induced cytochrome c release from the mitochondria, supporting published literature that Hsp27 blocks apoptosis by preventing downstream apoptosome activation.

Treatment of PDAC cells with an MK2 inhibitor, PF-3604422, alone was not toxic but when combined with SN38, significantly increased apoptosis (FIG. 2D). Consistent with MK2 knockdown, PF-3604422 abolished p-Hsp27 at baseline and following SN38 treatment (FIG. 2E). As further confirmation, these findings were replicated with another MK2 inhibitor, ATI-450, which selectively interacts with the p38MAPK:MK2 binding interface (FIGS. 2F, 2G).

To further understand the generalizability of activation of the MK2/Hsp27 pathway upon the mode of DNA damage, Pa01c and MIA paca-2 cells were treated with ultraviolet (UV) light. As expected, UV treatment robustly activated p38, MK2, and Hsp27, and p-Hsp27 was blocked by both MK2 inhibitors (FIG. 2H, 2I). Furthermore, treatment of non-pancreatic cancer cell lines (colon, gastric, breast) with combination SN38 and ATI-450 similarly sensitized the cells to apoptosis. Taken together, these data show that DNA damage, either via chemotherapy or UV irradiation, activates MK2 and subsequently Hsp27 as a pro-survival mechanism in diverse cellular contexts.

### SN38 Induces the MK2-Hsp27 Axis in a TAK1 and TNFα-Dependent Manner

To investigated the mechanism by which DNA damage activates MK2 and Hsp27, PDAC cells were treated with SN38 alone or in combination with inhibitors of IRAK4 (PF06650833), TLR9 (hydroxychloroquine, or HCQ), ATM (KU55933), ATR (VE821), and TAK1 (5z-7-oxozeanol). A previous study showed that DNA damage leads to activation of ATM, which then assembles cytosolic NEMO (IKKγ), RIP1, and TAK1, following which TAK1 can phosphorylate p38 and MK2. In addition, previous work in colon cancer showed that chemotherapy can induce TLR9 expression and activate IRAK4, which is a known upstream activator of p38

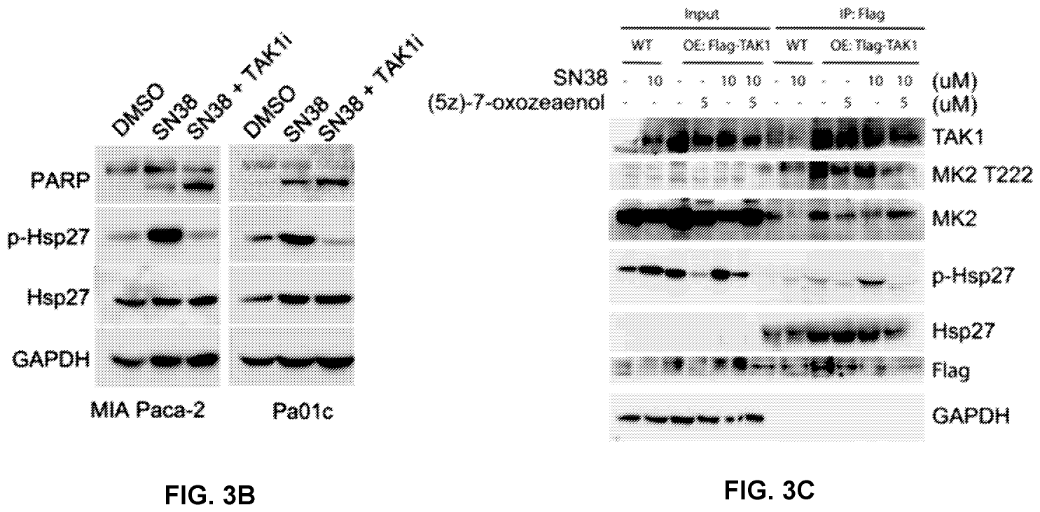

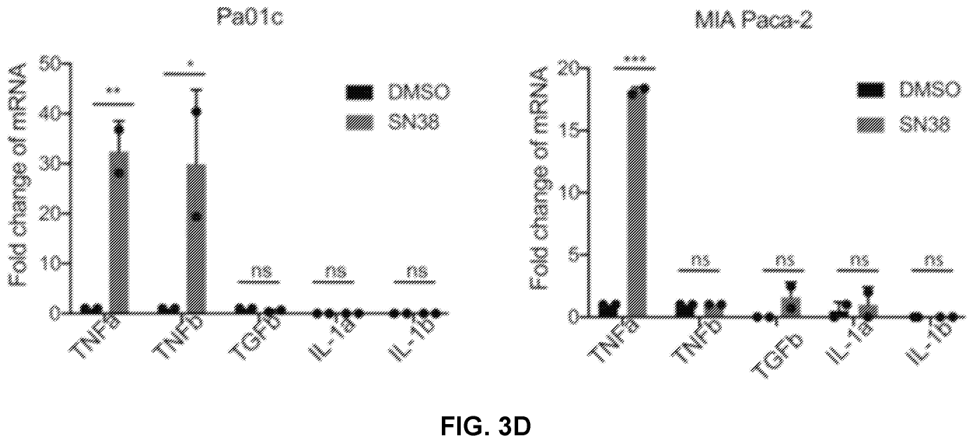

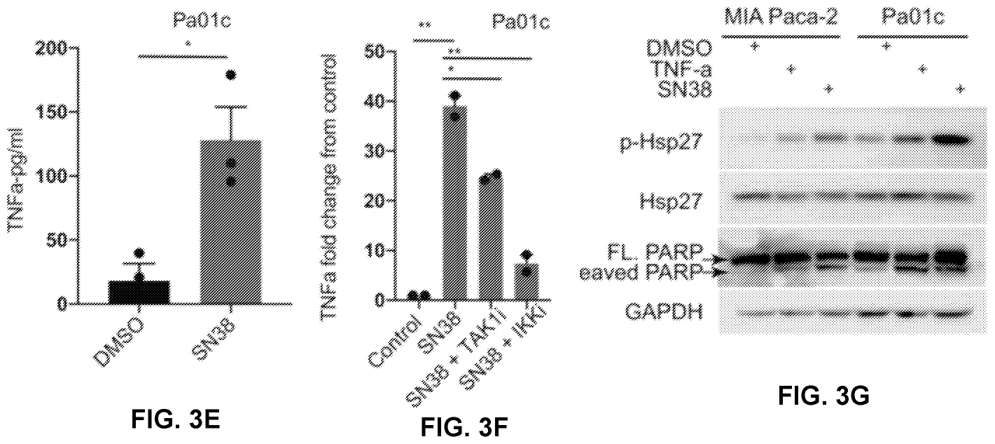

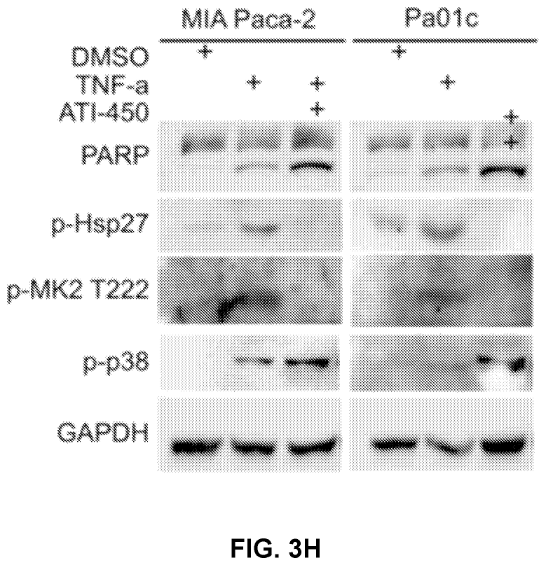

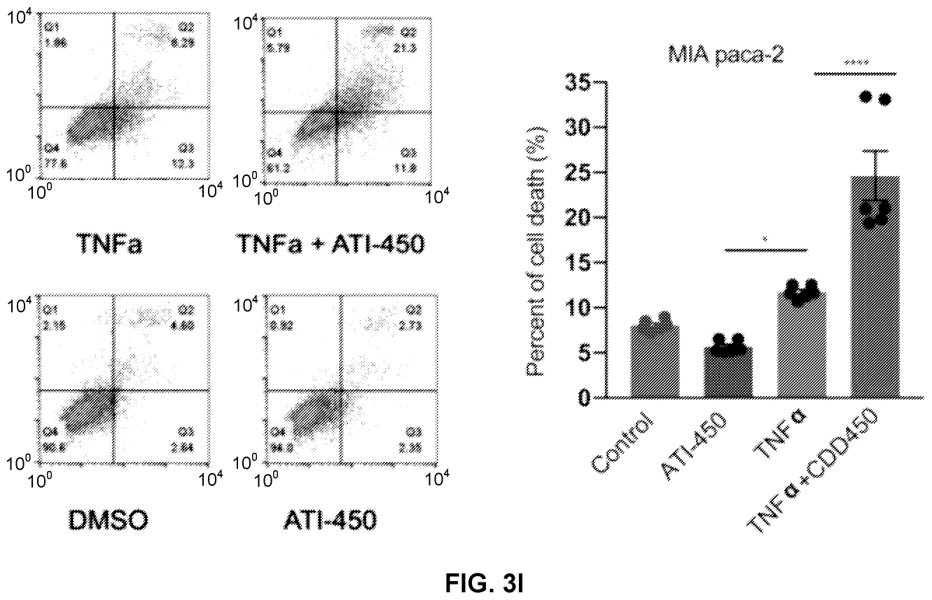

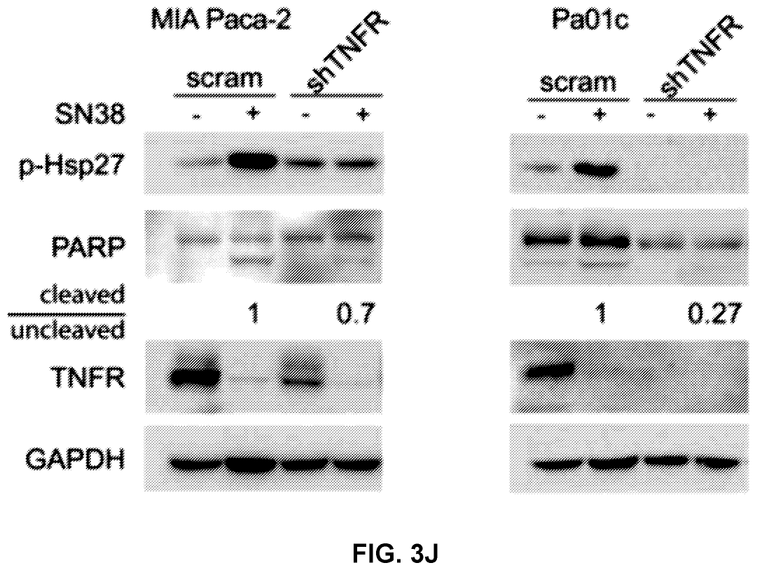

Of the inhibitors tested, only 5z-7-oxozeanol completely blocked SN38-induced p-p38, p-MK2, and p-Hsp27 (FIG. 3A) and potentiated the apoptotic effect of SN38 (FIG. 3B), strongly suggesting TAK1 as the upstream activating kinase of MK2 and Hsp27. As further support, treatment with SN38 significantly increased the binding of ectopically expressed flag-TAK1 with MK2 and Hsp27 (FIG. 3C). Contrary to previously published data, activation of p-Hsp27 by SN38 was not blocked by RIPK inhibitor (FIG. 3A), suggesting that TAK1 is engaged by a mechanism independent of intracellular ATM and RIPK and possibly through ligand-receptor interaction.

Because TAK1 is known to be a downstream kinase of TNF, TGFβ, and IL-1 receptors, a candidate approach was used to survey expression of TNFα/β, TGFβ, and IL-1α/β. In both MIA Paca-2 and Pa01c cells, TNFα mRNA expression was upregulated by ˜30 fold following SN38 treatment, although TNFβ was elevated in Pa01c but not MIA Paca-2 cells (FIG. 3D). Concordant with the qPCR findings, increased TNFα protein was detected in the supernatant of Pa01c cells by ELISA (FIG. 3E). Notably, SN38-induced induction of TNFα is abrogated by the IKK inhibitor IMD0354 and the TAK1 inhibitor 5z-7-oxozeanol (FIG. 3F), suggesting NF-κB and TAK1 activation as the triggering event for TNFα production.

Treatment of PDAC lines with recombinant TNFα upregulated p-Hsp27, but surprisingly also induced apoptosis as assayed by PARP cleavage and Annexin V staining (FIGS. 3F, 3G), suggesting that engagement of TNF receptor (TNFR) may have dual survival and pro-apoptotic effect. Because TNFR activates both RIPK and TAK1, the role of these two kinases in survival and activation of Hsp27 was investigated. Although TNFα treatment upregulated p-RIPK, treatment with RIPK inhibitor did not block p-Hsp27 or alter apoptosis. On the other hand, treatment with MK2 inhibitor ATI-450 abrogated TNFα-induced p-Hsp27 and augmented apoptosis (FIGS. 3H, 3I). Notably, autocrine TNFR activation is necessary for SN38-induced apoptosis, as silencing of TNFR attenuated SN38-induced PARP-cleavage as well as induction of p-Hsp27 (FIG. 3J). Interestingly, SN38 treatment resulted in decreased TNFR protein level, suggestive of endocytosis as would be seen with TNFα treatment. These data demonstrate that DNA damage induces NF-κB-dependent TNFα production, which had both pro-survival and pro-apoptotic functions. Inhibition of the pro-survival TAK1-MK2-Hsp27 axis shifted the balance towards apoptosis and thus may represent a promising therapeutic strategy.

### MK2 Upholds Chemotherapy-Induced Protective Autophagy

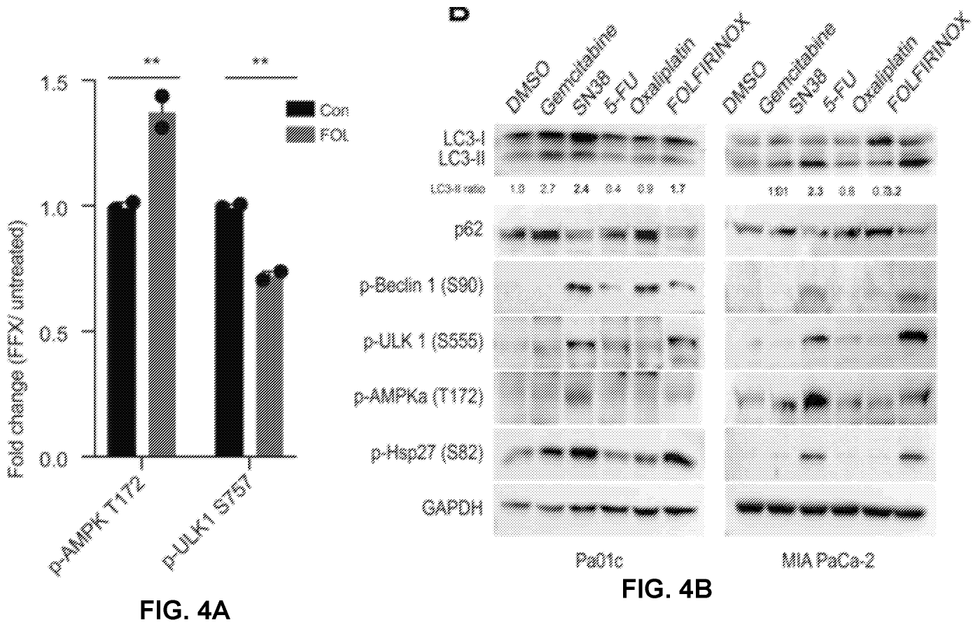

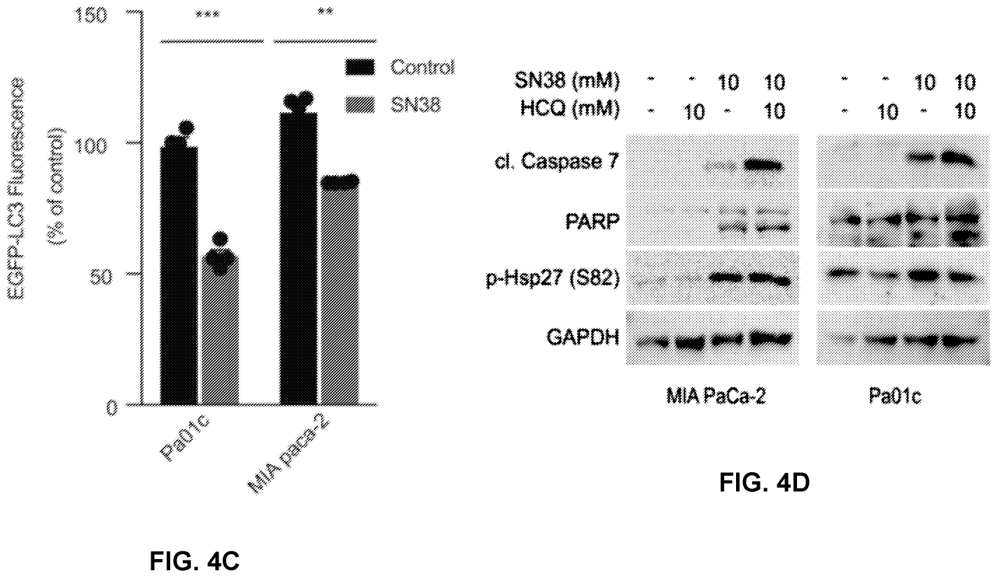

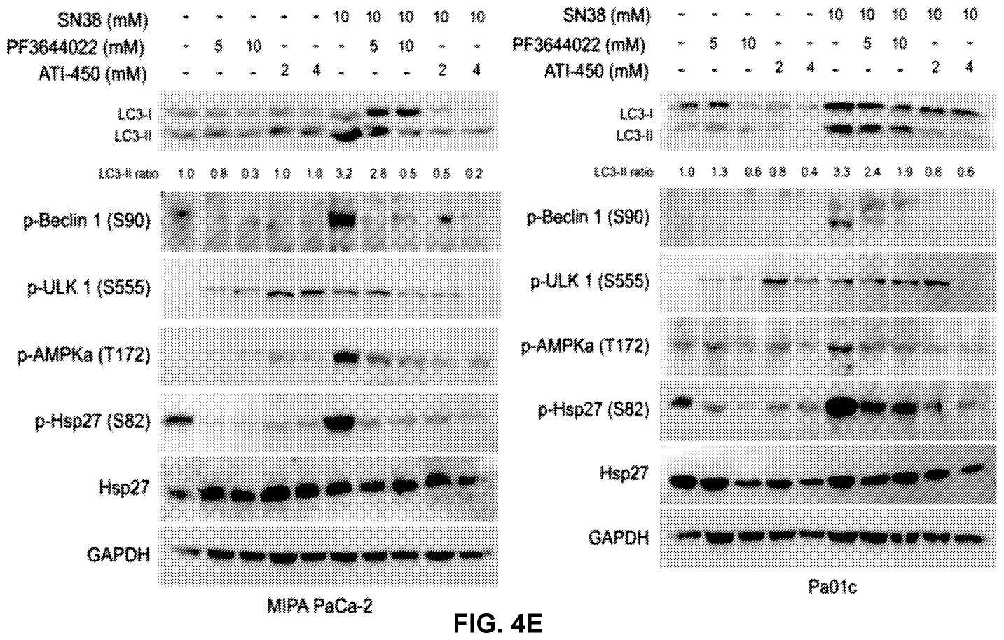

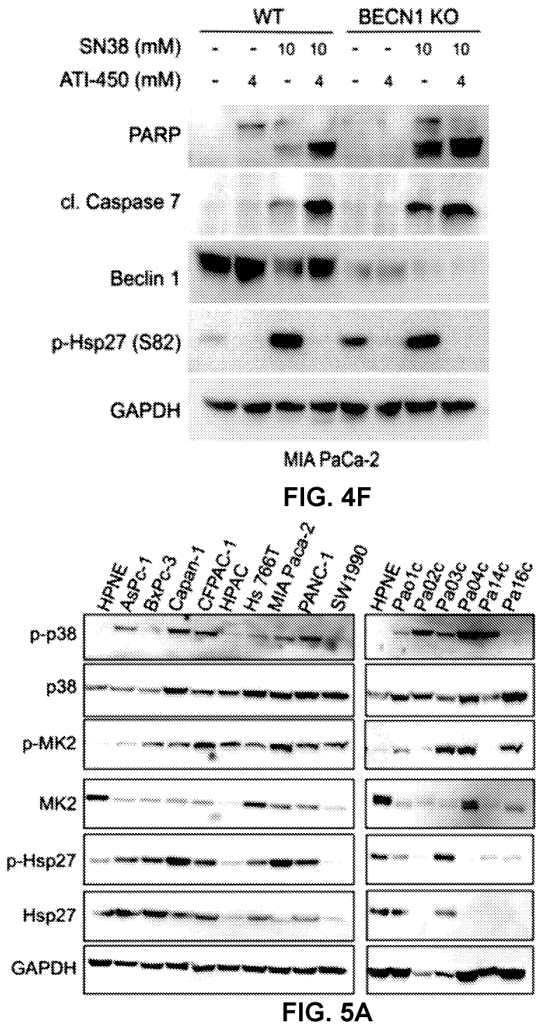

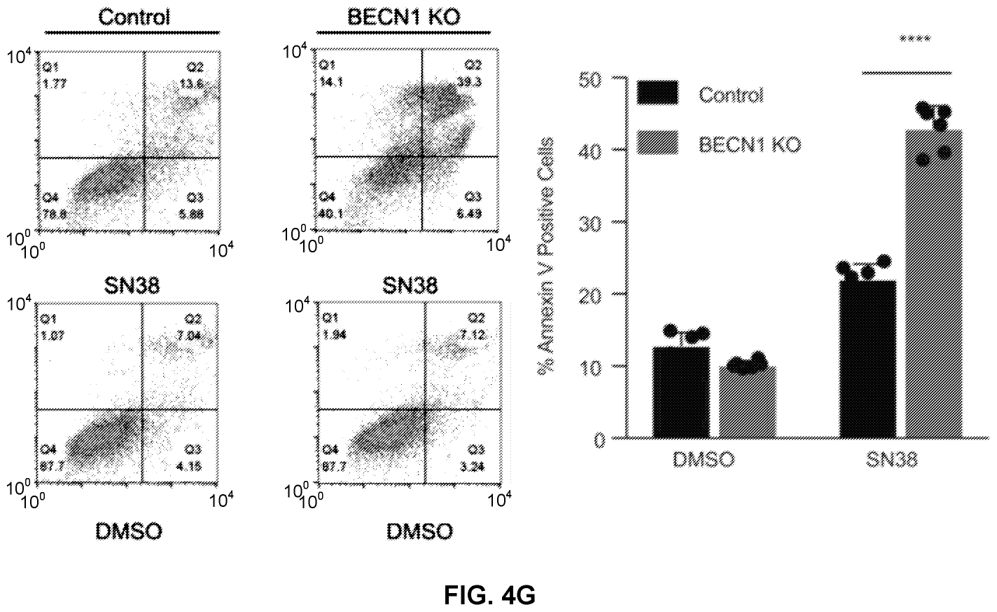

The RPPA analysis indicated decreased p-ULK1 at 5757 (inhibitory site) and increased p-AMPK at T172 (activation site), suggestive of increased autophagy upon FOLFIRINOX treatment (FIG. 4A). Chemotherapy, especially SN38, markedly induced autophagy markers observed using western blots that included increased p-ULK1 (S555), p-AMPKα (T172), p-Beclin1 (S90), as well as decreased p62 levels (FIG. 4B). SN38 also significantly decreased LC3-GFP reporter expression in PDAC cells (FIG. 4C), consistent with increased autophagy.

Increased autophagy is known to be part of DDR and can have multifaceted roles including the promotion of DNA repair and hence cellular survival, induction of senescence, and cell death. PDAC cells treated with SN38 and hydroxychloroquine (HCQ), a commonly used autophagy inhibitor, were found to have significantly higher levels of apoptosis, suggesting that DNA damage-induced autophagy is cytoprotective (FIG. 4D).

Previously published research showed that MK2/MK3 activates starvation-induced autophagy by phosphorylating Beclin-1. MK2 inhibitors PF-3644022 and ATI-450 were both found to robustly suppress SN38-induced p-Beclin1 and LC3-II, supporting the hypothesis that MK2 was a positive regulator of Beclin-1 and autophagy during DNA damage (FIG. 4E). Ablation of Beclin-1 by CRISPR/Cas9 also sensitized PDAC cells to SN38-induced apoptosis, and the addition of ATI-450 only marginally increased apoptosis in Beclin-1-ablated cells (FIGS. 4F, 4G). However, Hsp27 knockdown in PDAC cells did not significantly affect SN38 induced activation of autophagy markers, suggesting that activation of Hsp27 and autophagy were parallel downstream effects of MK2. Taken together, these findings suggest that targeting MK2 suppresses chemotherapy-induced activation of protective autophagy.

### MK2 is Activated and Required for PDAC Tumorigenesis in KPPC Mice

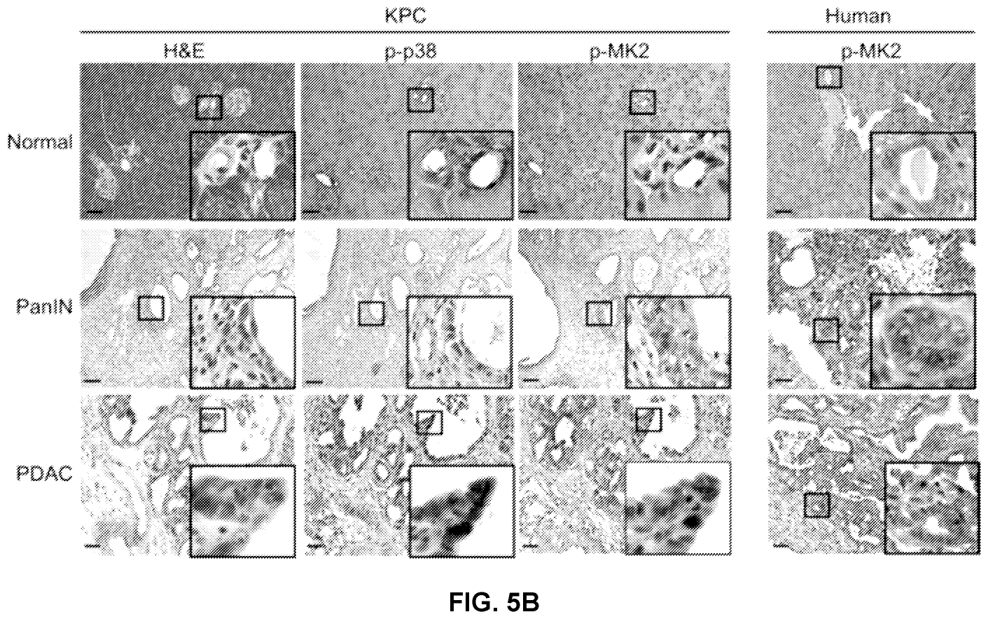

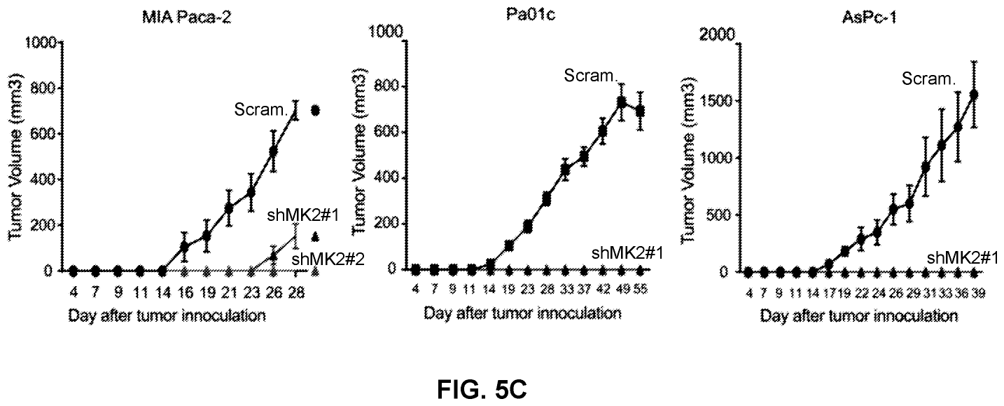

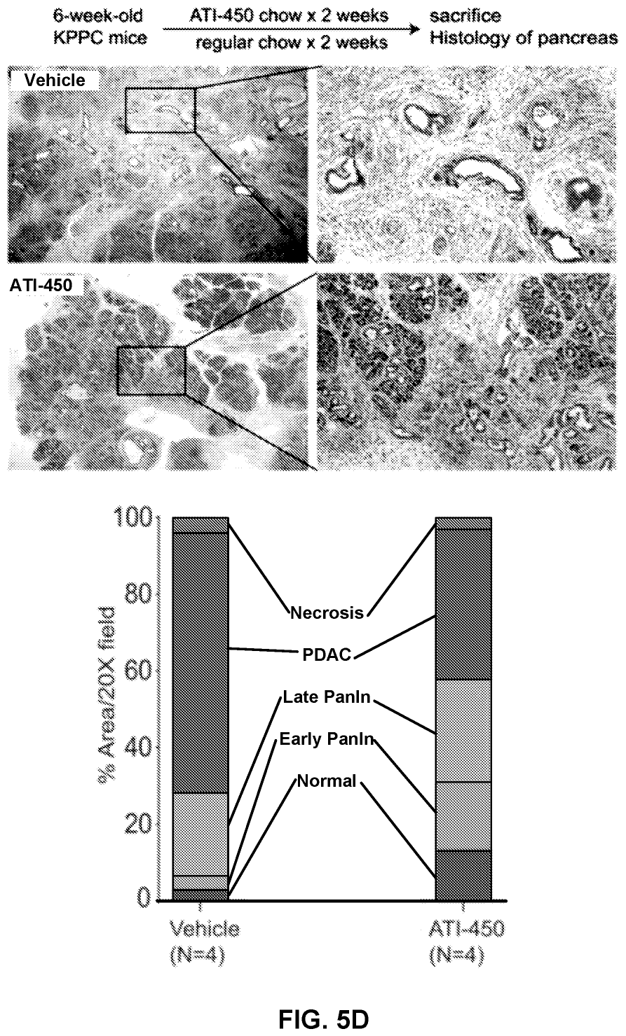

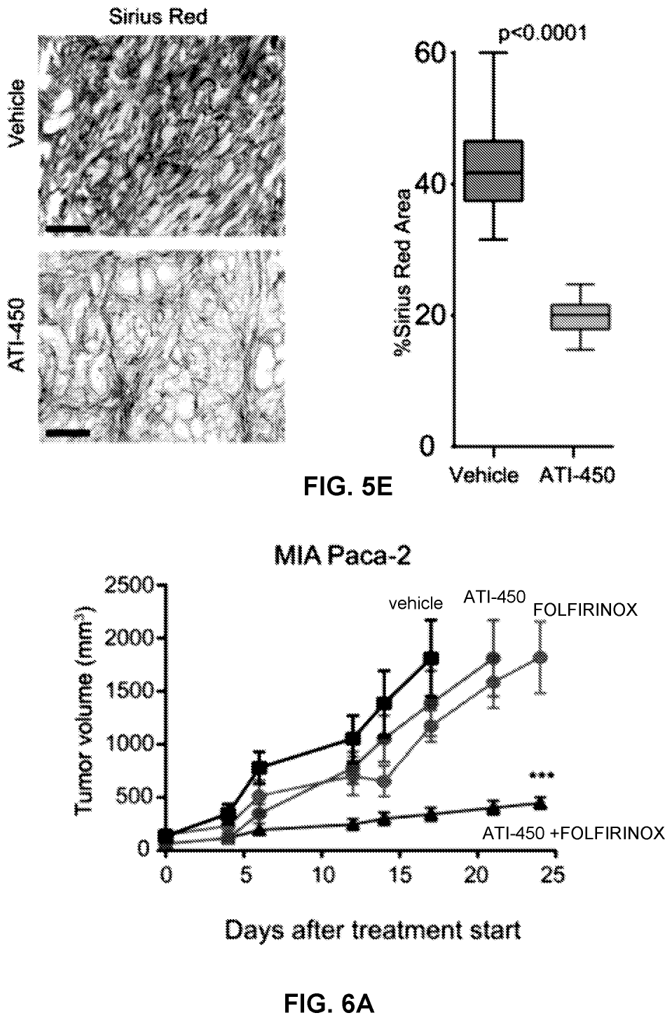

Besides genotoxic stress, during stepwise neoplastic progression PDAC cells must endure other adverse conditions including replication stress, starvation, hypoxia, and anchorage-independent growth. Enhanced MK2-Hsp27 signaling may provide a survival advantage for PDAC cells. The activation status of p38, MK2, and Hsp27 was surveyed in a panel of PDAC lines, using an immortalized human pancreatic normal epithelial (HPNE) line as normal control. p38, MK2, and Hsp27 were found to be activated by phosphorylation, to various degrees, in most PDAC lines (FIG. 5A). Increased p-MK2 immunohistochemical (IHC) staining intensity was also observed during progression from normal to precancerous PanIN lesions and invasive PDAC in mouse and human tissues (FIG. 5B). Silencing of MK2 or Hsp27 completely abolished the anchorage-independent growth of PDAC lines in soft agar, and loss of MK2 completely blocked the in vivo tumorigenicity of three different PDAC lines which had low (AsPc-1), medium (Pa01c), and high (MIA Paca-2) p-MK2 levels, respectively (FIG. 5C).

To complement the human xenograft models, which lacked the desmoplastic tumor microenvironment and intact immune system essential for PDAC development, an autochthonous KPPC (p48-Cre; p53flox/flox; LSL-KRASG12D) mouse model was treated and observed. KPPC mice were treated with vehicle or ATI-450 ad libitum in chow starting from 6 weeks-old of age when foci of invasive PDAC start to appear. After two weeks of treatment, all mice were euthanized and their pancreata were subjected to histologic analysis. ATI-450-treated pancreata were found to have significantly lower PDAC burdens and relatively higher fractions of normal or early PanIN lesions (FIG. 5E), indicating a delay in neoplastic progression. Accordingly, ATI-450-treated tumors were significantly less fibrotic, as determined by Sirius Red staining of tumor sections (FIG. 5F). These results indicated that MK2 has an important role not only in tumorigenesis of established PDAC cells but also during stepwise development of PDAC, and thus is a promising therapeutic target.

### MK2 Inhibition Augments the Efficacy of Chemotherapy in Human PDAC Model and KPPC Mice

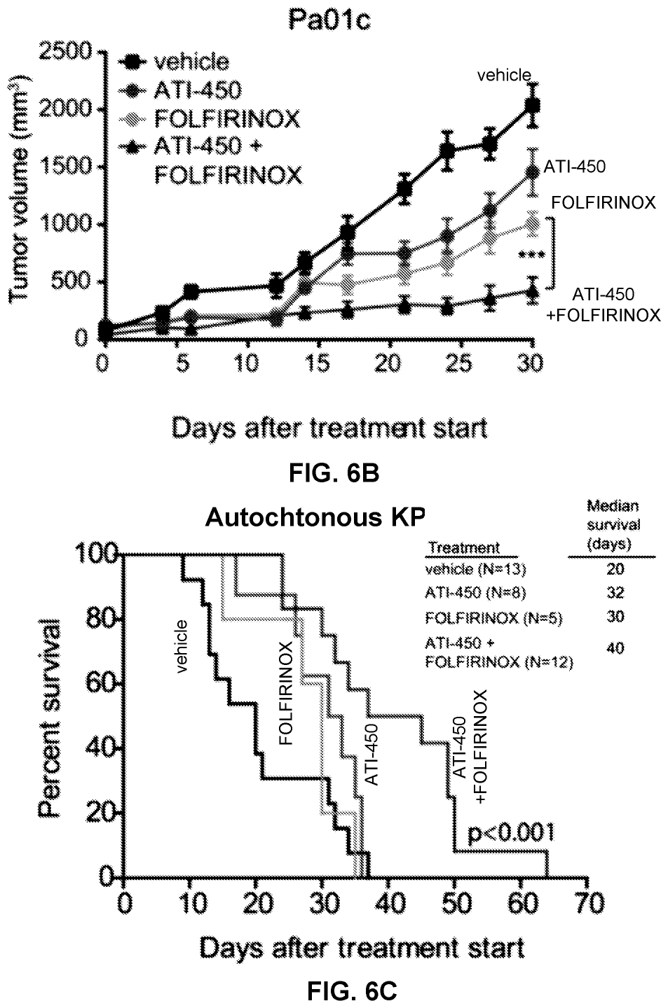

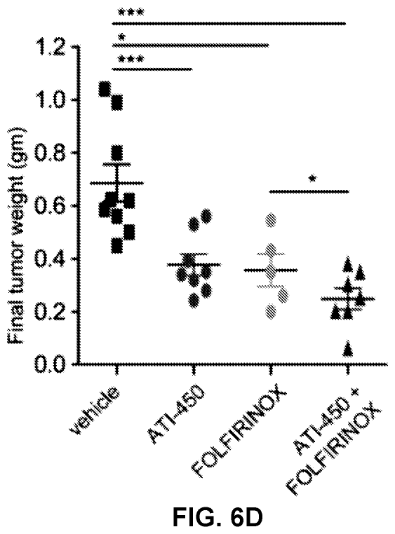

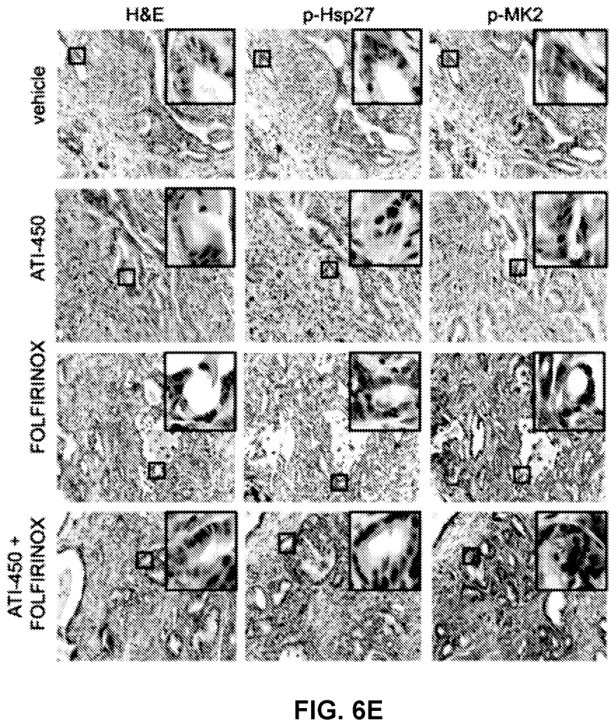

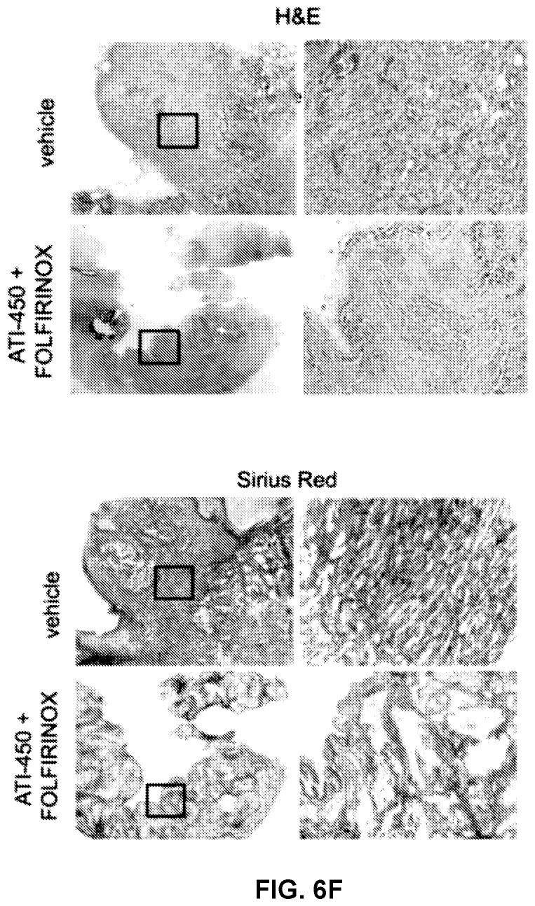

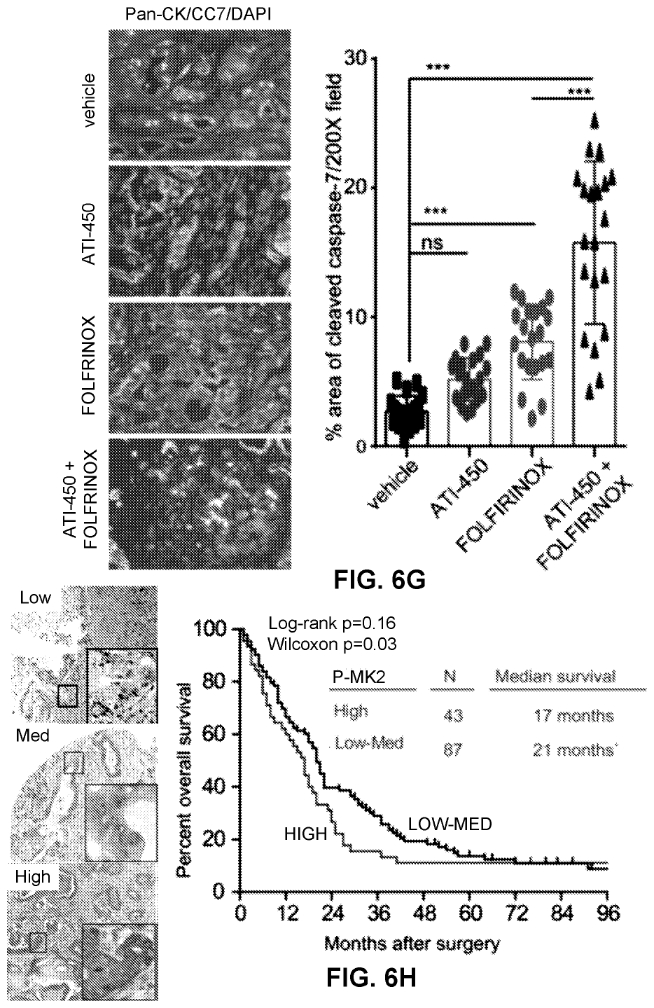

To test the therapeutic efficacy of the MK2 inhibitor, ATI-450, in combination with FOLFIRINOX in preclinical human and mouse models, the following experiments were conducted. Nude mice bearing subcutaneous human MIA Paca-2 and Pa01c tumors greater than 50 mm3 were treated with vehicle, ATI-450 in chow, weekly FOLFIRINOX intraperitoneally, or the combination of ATI-450 and FOLFIRINOX. In both models, the ATI-450/FOLFIRINOX combination was significantly more efficacious in suppressing tumor growth than either treatment alone (FIGS. 6A, 6B).

Because the dense stroma and immune system also contributed to treatment resistance, the autochthonous KPPC model was similarly treated and observed. All mice were treated beginning at 6 weeks of age until humane endpoints were reached. While ATI-450 or FOLFIRINOX both improved survival significantly greater than vehicle, the combination of ATI-450 and FOLFIRINOX was significantly more efficacious (FIG. 6C). Tumors taken from end-stage mice treated with ATI-450/FOLFIRINOX combination were significantly smaller (FIG. 6D). Immunohistochemical analyses of tumor sections showed upregulated p-MK2 and p-Hsp27 staining intensity in FOLFIRINOX-treated tumors, and p-Hsp27 was completely suppressed by ATI-450 (FIG. 6E), demonstrating in vivo on-target effect of ATI-450. Compared to ATI-450 or FOLFIRINOX-treated tumors, which were smaller but retained a significant fraction of overt PDAC, combo-treated tumors showed vast areas of necrosis with scattered foci of PDAC (FIG. 6F, upper panels). Collagen fibrils in combo-treated tumors were thin and sparse as opposed to the thick and dense appearance seen in vehicle-treated tumors (FIG. 6F, lower panels). Combo-treated tumors had significantly less cytokeratin-positive ductal epithelia, and a significantly higher number of cleaved-caspase-7 positive cells (FIG. 6G), suggesting the destruction of neoplastic epithelia by ATI-450/FOLFIRINOX.

The prognostic impact of p-MK2 was investigated in a clinically annotated PDAC tissue microarray constructed from surgical specimens of PDAC patients. The staining intensity of p-MK2 was calculated using a composite score of staining intensity and distribution obtained using an automated slide scanner. From 130 cases analyzed, high expression of p-MK2 in PDAC specimens was associated with poorer overall survival, compared to patients with low and medium p-MK2 staining (P=0.03) based on Wilcoxon, a statistical test that gives more weight to early deaths, as is typically seen with PDAC patients. Overall, these results strongly support targeting MK2 as a strategy to augment the therapeutic efficacy of FOLFIRINOX or other irinotecan-containing chemotherapy, such as 5-FU/liposomal irinotecan, in clinical trials for PDAC patients.

### Discussion

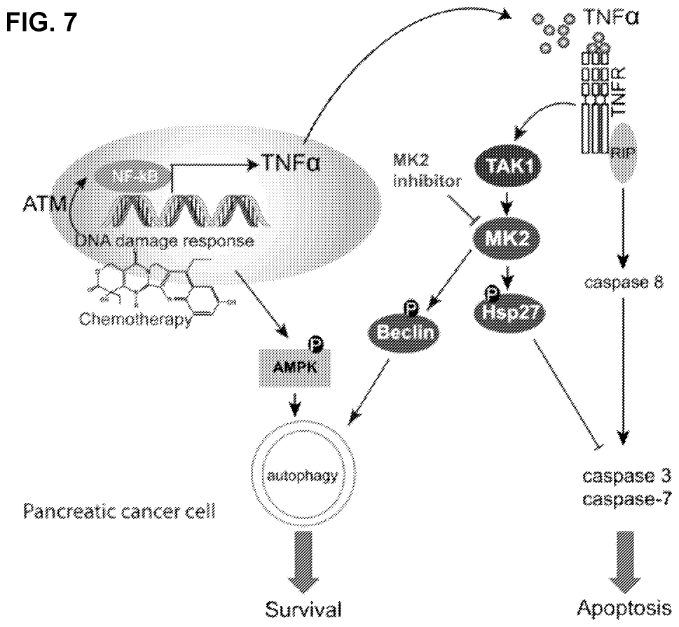

Chemotherapy including FOLFIRNOX and gemcitabine/nab-paclitaxel remains the cornerstone for PDAC treatment and contributes to the recent improvement in the 5-year survival of PDAC. 5-FU plus liposomal irinotecan is currently the only FDA-approved 2nd line regimen for patients who have failed gemcitabine-based chemotherapy. Targeted and immuno-therapies remain largely ineffective and have been the focus of intensive research. In parallel, improving the efficacy of current chemotherapy regimens represents a practical approach that could immediately improve the outcome of PDAC patients. The experiments described above may be the first to investigate resistance mechanisms to FOLFIRINOX in PDAC, whereas most studies have focused on gemcitabine. Using an unbiased RPPA approach, the MK2-Hsp27 axis was shown to be a major survival mechanism that protects PDAC cells from FOLFIRINOX. Mechanistically, DNA-damage was caused by FOLFIRINOX treatment that activates the NF-κB pathway and production of TNFα. Autocrine engagement of the TNFR triggers both apoptotic and the TAK1-MK2-Hsp27 survival pathway. Targeting MK2 inactivates pro-survival Hsp27 and simultaneously blocks Beclin-1 from facilitating protective autophagy, both of which tip the balance towards apoptosis (FIG. 7).

Literature on the role of MK2-Hsp27 in gemcitabine-induced apoptosis is controversial. Gemcitabine was shown to activate the p38-MK2-Hsp27 stress response cascade, which was also observed in these experiments but at a much lower degree than SN38 or FOLFIRINOX. Gemcitabine-resistant PDAC tissues express a higher level of Hsp27 and p-Hsp27, indicating that targeting Hsp27 could be beneficial. On the contrary, others have shown that overexpression of Hsp27 sensitizes PDAC cells to gemcitabine. In a randomized phase II clinical trial, the addition of Apatorsen, an antisense oligonucleotide against Hsp27, to gemcitabine/nab-paclitaxel failed to improve the outcomes of patients with metastatic PDAC. Similarly, the consequence of inhibiting MK2 is highly context-dependent, i.e. may vary with the class of chemotherapeutics used and tumor cells examined. MK2 inhibition has been previously shown to reduce gemcitabine-induced DNA replication stall and to later facilitate the restoration of DNA replication in PDAC cells, and therefore to attenuate the efficacy of gemcitabine. On the other hand, p38/MK2 signaling was proposed as a third checkpoint response pathway, in parallel to the ATM/Chk2 and ATR/Chk1 cascades, following more severe DNA damage as incurred by cisplatin or topoisomerase inhibitors. In this setting, MK2 phosphorylates hnRNPA0, TIAR, and PARN, which stabilizes multiple RNA transcripts essential for DNA damage response. The results of the experiments described above using FOLFIRINOX, which causes more severe DNA damage, are in line with these latter studies and support inhibiting MK2 to deepen FOLFIRINOX-induced apoptosis.

Besides Hsp27, the results of the experiments described above showed that targeting MK2 additionally blocks Beclin-1 activation, thereby abrogating protective autophagy. Autophagy, or self-eating, is commonly adopted by cancer cells as a means to acquire macromolecules necessary for growth and proliferation, especially when extracellular nutrients are scarce. In fact, starvation activates Beclin-1 and autophagy in an MK2-dependent manner in MCF7 breast cancer and U2OS osteosarcoma cell lines. In an autochthonous PDAC mouse model, PDX1-Cre mediated deletion of ATG5 results in increased tumor initiation but impedes the progression to PDAC. In support, induced expression of dominant negative ATG4BC74A in neoplastic cells or host (stromal) cells to block autophagy significantly impaired PDAC development and growth of PDAC lines in vivo. Furthermore, pharmacologic inhibition of MEK or ERK was counteracted by increased protective autophagy, which when inhibited augments the anti-tumor effect of MEK or ERK inhibitors. Together, these studies support inhibiting autophagy as a therapeutic approach in PDAC. The role of chemotherapy-induced autophagy is poorly described in PDAC. The results of the experiments described above showed that FOLFIRINOX potently induces autophagy, and co-treatment with HCQ or ablation of Beclin-1 augmented apoptosis, demonstrating that autophagy is cytoprotective. However, an effective autophagy inhibitor is elusive. HCQ blocks autophagy by neutralizing lysosomal pH and Toll-like receptor signaling. However, the pharmacodynamic effect of HCQ in human patients is highly inconsistent. In a phase II clinical study, the addition of HCQ failed to improve the survival benefit of gemcitabine/nab-paclitaxel, although patients who received HCQ plus chemotherapy had higher response rates. These studies underscore the importance of developing more effective and reliable autophagy inhibitors. To this end, MK2 inhibition blocks chemotherapy-induced autophagy markers Beclin-1, AMPKα, and ULK1, and therefore represents an effective approach to block autophagy.

The MK2-Hsp27 pathway can be activated as a downstream event of ATM/ATR checkpoints. However, neither ATM nor ATR inhibitors could suppress SN38-induced MK2 and Hsp27. Instead, these proteins were activated by engagement of autocrine TNFα secretion and activation of TNFR and TAK1. Importantly, SN38-induced TNFα secretion mediates both survival and death in PDAC cells. While TNFα treatment alone induces apoptosis, silencing TNFR attenuates the apoptotic effect of SN38, suggesting that the full cytotoxic effect of SN38 is exerted not only through direct DNA damage but also the apoptotic machinery incurred by TNFR signaling. As opposed to the TAK1-MK2 axis, which is pro-survival, targeting RIPK, another kinase downstream of TNFR, did not potentiate the apoptotic effect of SN38. Therefore, by manipulating the downstream pathways, the effect of TNFα signaling can be harnessed for therapeutic purposes.

Besides Hsp27, the RPPA described above also identified c-JUN and ERK to be significantly phosphorylated upon FOLFIRINOX treatment. Targeting MEK and ERK, both downstream effectors of KRAS oncoproteins, has been clinically challenging due to the emergence of multiple escape mechanisms. The addition of the MEK inhibitor trametinib failed to potentiate the effect of gemcitabine in a clinical trial. Therefore, successful targeting of the MAPK pathway requires the development of combinatorial regimens that can effectively and durably curb these escape mechanisms. Inhibition of JNK and hence phosphorylation of c-Jun was shown to promote 5-FU and gemcitabine-induced production of intracellular reactive oxygen species and hence apoptosis, providing the rationale for combining a JNK inhibitor with chemotherapy for PDAC patients. Paradoxically, deletion of MKK4, the upstream activator of JNK, markedly accelerates KRASG12D-driven PDAC development in a genetic mouse model, raising concern for a potential detrimental effect with JNK inhibitors. JNK inhibition was found to attenuate the apoptotic effect of FOLFIRINOX. In contrast, the results described above show that MK2 inhibition has therapeutic potential in PDAC. As a single agent, ATI-450 significantly delayed the progression of PDAC in KPPC mice, a highly aggressive autochthonous PDAC mouse model. Even more strikingly, the addition of ATI-450 to FOLFIRINOX almost completely ablated all PDAC lesions in KPPC mice, an observation that has not been reported in the literature. Because all p48-positive cells undergo an acinar-to-ductal transformation and neoplastic progression in this model, all combo-treated mice were believed to eventually die from pancreatic insufficiency. MK2 knockout mice are viable and have a normal lifespan, although they are resistant to lipopolysaccharide-induced endotoxic shock. ATI-450 is currently being tested in a phase 1/2 clinical trial for patients with rheumatoid arthritis and is well-tolerated.

The results of these experiments revealed several new insights into the survival signaling of PDAC cells treated with FOLFIRINOX (a cocktail of 5-FU, SN38 which is the active metabolite of irinotecan, and oxaliplatin). Activation of heat shock protein 27 (Hsp27), driven by the TNFα-TAK1-p38-MK2 axis, was found to be a major survival mechanism that enables PDAC cells to tolerate FOLFIRINOX. The results of these experiments further provided preclinical evidence, using patient-derived cell lines (PDCL) and an autochthonous PDAC mouse model (p48-Cre;p53flox/flox; LSL-KRASG12D, termed KPPC mice), that the MK2 inhibitor, ATI-450, significantly potentiated the cytotoxic effect of FOLFIRINOX and prolonged the survival of KPPC mice. Taken together, the results of these studies demonstrate the potential efficacy of MK2 inhibition in combination with irinotecan-containing chemotherapy for the treatment of pancreatic ductal adenocarcinoma.

### Example 2: Evaluation of MK2 as a Therapeutic Target in Pancreatic Cancer

To assess the preclinical efficacy of ATI-450 in PDAC models, the following experiments will be conducted.

NOD-SCID mice will be inoculated subcutaneously at bilateral flanks with MIA Paca-2 (a conventional PDAC line) and Pa01c (an early-passaged patient-derived PDAC line) cells. When tumors have achieved 100 mm³ in volume, mice will be randomized to receive vehicle, chemotherapy (FOLFOX), ATI-450 (chow), or chemotherapy plus chow. Serial measurements of tumor volume will be recorded, and each mouse will be taken down when its tumor has reached 2500 mm³.

Cytokine array analyses will be performed on conditioned media collected from two different PDAC CAFs (CAF1 and CAF2) with and without MK2 knockdown by shRNA that will be treated with DMSO, ATI-450 for 24 hours. Standard in vitro functional studies including collagen-contraction, survival, migration, and co-culture assays with PDAC (MIA Paca-2) cells will also be performed. PDAC:CAF mixture co-injection will be performed on nude mice to confirm the role of CAF MK2 in promoting PDAC tumorigenesis and fibrosis.

IHC analyses will be performed on tumor samples collected from KPPC mice treated with vehicle, ATI-450, and/or gemcitabine/anti-PD1/anti-CTLA4 cocktail to determine how ATI-450 affects the abundance of CD4, CD8, CD206 macrophage, CD4/Treg cells, CD103 dendritic cells as well as the degree of fibrosis by Sirius Red. Using a transplantable KPC model, ATI-450 in combination with FOLFOX/CD40 agonist/anti-PD1, or the FOLFOX/anti-CTLA4/anti-PD1 cocktail will be similarly tested.

What is claimed is:

1. A composition for the treatment of pancreatic cancer in a subject in need, the composition comprising an effective amount of an MK2 inhibiting agent and an effective amount of a chemotherapy composition.

2. The composition of claim 1, wherein the MK2 inhibiting agent is selected from a small molecule, an interfering protein, an antibody, an shRNA, and an siRNA.

3. The composition claim 1, wherein the MK2 inhibiting agent targets MK2 or MK2R1.

4. The composition claim 2, wherein the MK2 inhibiting agent is PF-3644022 or ATI-450.

5. The composition of claim 1, wherein the chemotherapy composition comprises irinotecan.

6. The composition of claim 1, wherein the chemotherapy composition comprises at least one of leucovorin calcium (folinic acid), fluorouracil (5-FU), irinotecan, oxaliplatin, gemcitabine, nab-paclitaxel, and any combination thereof.

7. The composition of claim 6, wherein the chemotherapy composition is selected from FOLFIRINOX, FOLFOX, FOLFOX/CD40 agonist/anti-PD1 cocktail, FOLFOX/anti-CTLA4/anti-PD1 cocktail, and gemcitabine/nab-paclitaxel.

8. A method for treating pancreatic cancer in a subject in need, the method comprising administering an effective amount of an MK2 inhibiting agent to the subject.

9. The method of claim 8, further comprising administering an effective amount of a chemotherapy composition in combination with the effective amount of the MK2 inhibiting agent.

10. The method of claim 8, wherein the MK2 inhibiting agent is selected from a small molecule, an interfering protein, an antibody, an shRNA, and an siRNA.

11. The method of claim 8, wherein the MK2 inhibiting agent targets MK2 or MK2R1.

12. The method of claim 8, wherein the MK2 inhibiting agent is PF-3644022 or ATI-450.

13. The method of claim 9, wherein the chemotherapy composition comprises irinotecan.

14. The method of claim 9, wherein the chemotherapy composition comprises at least one of leucovorin calcium (folinic acid), fluorouracil (5-FU), irinotecan, oxaliplatin, gemcitabine, nab-paclitaxel, and any combination thereof.

15. The method of claim 9, wherein the chemotherapy composition is selected from OLFIRINOX, FOLFOX, FOLFOX/CD40 agonist/anti-PD1 cocktail, FOLFOX/anti-CTLA4/anti-PD1 cocktail, and gemcitabine/nab-paclitaxel.

16. The method of claim 8, wherein the pancreatic cancer is pancreatic ductal adenocarcinoma.
